(19) United States
(12) Patent Application Publication
(10) Pub. No.: US 2019/0304574 A1
(43) Pub. Date: Oct. 3, 2019

(54) SYSTEMS AND METHODS FOR MANAGING SERVER-BASED PATIENT CENTRIC MEDICAL DATA

(71) Applicant: Richard A. Weinstock, Palm Beach Gardens, FL (US)

(72) Inventors: Richard A. Weinstock, Palm Beach Gardens, FL (US); Stephen K. Mansfield, Wellington, FL (US)

(21) Appl. No.: 15/938,215

(22) Filed: Mar. 28, 2018

(51) Int. Cl. G16H 10/20; G16H 10/60; H04L 9/06

(52) U.S. Cl. CPC G16H 10/20; H04L 9/0637; G16H 10/60

(57) ABSTRACT

Systems, apparatuses, methods, and computer program products are disclosed for managing server-based patient centric medical data (PCMD). An example method includes transmitting a first template to a first computing device and transmitting a second template to a second computing device. The example method further includes receiving first patient data provided in response to the first template and receiving second patient data provided in response to the second template. The example method further includes generating a first record based on the first template, the first patient data, the second template, and the second patient data. The example method further includes generating a second record based on the second template and the second patient data.

### TECHNOLOGICAL FIELD

Example embodiments of the present disclosure relate generally to data management and, more particularly, to systems and methods for managing medical data.

### BACKGROUND

The inventors have discovered problems with existing mechanisms for managing medical data. Through applied effort, ingenuity, and innovation, the inventors have solved many of these identified problems by developing solutions embodied by the present disclosure and described in detail below.

### BRIEF SUMMARY

Systems, apparatuses, methods, and computer program products are disclosed herein for managing server-based patient centric medical data (PCMD) using a PCMD system that comprises template circuitry and records circuitry. The PCMD system provided herein solves the above problems by centralizing all patient data in the PCMD system.

In one example embodiment, a system is provided for managing PCMD. The system may comprise circuitry configured to transmit a patient life medical template (PLMT) for display to a first computing device; receive first patient data provided as input to the PLMT via the first computing device; generate a first record based on the first patient data provided as input to the PLMT via the first computing device; transmit a physician patient medical template (PPMT) for display to a second computing device; receive second patient data provided as input to the PPMT via the second computing device; and generate a second record based on the second patient data provided as input to the PLMT via the second computing device.

In another example embodiment, an apparatus is provided for managing PCMD. The apparatus may comprise circuitry configured to transmit a PLMT for display to a first computing device; receive first patient data provided as input to the PLMT via the first computing device; generate a first record based on the first patient data provided as input to the PLMT via the first computing device; transmit a PPMT for display to a second computing device; receive second patient data provided as input to the PPMT via the second computing device; and generate a second record based on the second patient data provided as input to the PLMT via the second computing device.

In yet another example embodiment, a method is provided for managing PCMD. The method may comprise transmitting a PLMT for display to a first computing device; receiving first patient data provided as input to the PLMT via the first computing device; generating a first record based on the first patient data provided as input to the PLMT via the first computing device; transmitting a PPMT for display to a second computing device; receiving second patient data provided as input to the PPMT via the second computing device; and generating a second record based on the second patient data provided as input to the PLMT via the second computing device.

In yet another example embodiment, a computer program product is provided for managing PCMD. The computer program product may comprise at least one non-transitory computer-readable storage medium having computer-executable program code stored therein. The computer-executable program code may comprise program code instructions configured to transmit a PLMT for display to a first computing device; receive first patient data provided as input to the PLMT via the first computing device; generate a first record based on the first patient data provided as input to the PLMT via the first computing device; transmit a PPMT for display to a second computing device; receive second patient data provided as input to the PPMT via the second computing device; and generate a second record based on the second patient data provided as input to the PLMT via the second computing device.

The foregoing brief summary is provided merely for purposes of summarizing some example embodiments illustrating some aspects of the present disclosure. Accordingly, it will be appreciated that the above-described embodiments are merely examples and should not be construed to narrow the scope of the present disclosure in any way. It will be appreciated that the scope of the present disclosure encompasses many potential embodiments in addition to those summarized herein, some of which will be described in further detail below.

### BRIEF DESCRIPTION OF THE FIGURES

The accompanying figures, which are not necessarily drawn to scale, illustrate embodiments and features of the present disclosure. Together with the specification, including the brief summary above and the detailed description below, the accompanying figures serve to explain the embodiments and features of the present disclosure. The components illustrated in the figures represent components that may or may not be present in various embodiments or features of the disclosure described herein. Accordingly, some embodiments or features of the present disclosure may include fewer or more components than those shown in the figures while not departing from the scope of the disclosure.

FIG. 1 illustrates a system diagram of a set of devices that may be involved in some example embodiments described herein;

FIG. 2 illustrates a schematic block diagram of example circuitry that may perform various operations in accordance with some example embodiments described herein;

FIG. 3 illustrates a system diagram of a set of devices that may be involved in providing server-based patient centric medical data (PCMD) in accordance with some example embodiments described herein;

FIG. 4 illustrates a system diagram of a set of devices that may be involved in providing a PCMD software as a service (SaaS) cloud server in accordance with some example embodiments described herein;

FIG. 5 illustrates a schematic diagram for providing patient secure credentials in accordance with some example embodiments described herein;

FIG. 6 illustrates a schematic diagram for providing physician secure credentials in accordance with some example embodiments described herein;

FIG. 7 illustrates a schematic diagram for providing patient and physician secure access in accordance with some example embodiments described herein;

FIG. 8 illustrates a token PCMD sequence diagram in accordance with some example embodiments described herein;

FIG. 9 illustrates a system diagram of a set of devices that may be involved in providing a patient PCMD application in accordance with some example embodiments described herein;

FIG. 10 illustrates a system diagram of a set of devices that may be involved in providing a physician PCMD application in accordance with some example embodiments described herein;

FIG. 11 illustrates a system diagram of a set of devices that may be involved in providing a server-based PCMD application architecture in accordance with some example embodiments described herein; and

FIG. 12 illustrates an example flowchart for managing server-based PCMD in accordance with some example embodiments described herein.

### DETAILED DESCRIPTION

Some embodiments of the present disclosure will now be described more fully hereinafter with reference to the accompanying figures, in which some, but not all embodiments of the disclosures are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

### Overview

Advances in medical technology, diagnosis, and treatment has dramatically advanced over the last couple of decades. These advances have also dramatically increased the amount of patient medical data, including X-rays, computerized tomography (CT or CAT) scans, electrocardiograms (ECGs or EKGs), biopsies, blood tests, bodily fluid tests, and many more. This patient medical data often is stored in many sources and scattered between multiple physicians' offices, hospitals, medical centers, clinics, pharmacies, and medical test facilities. Today there is no common method or interface to access this data. Government and industry regulations also make access to this data problematic if not impossible. As a result, medical professionals do not have access to the data they need to make meaningful diagnosis and treatment. Physician access to this data, especially historical data, is often difficult and sometimes impossible to retrieve. Thus, the patient's physician often does not have easy access or even awareness of the patient's medical data. Easy access to this data may help physicians make better diagnosis and treatment decisions for their patients.

In an attempt to facilitate access to patient medical data, several centralized patient medical data systems have been proposed. However, these conventional systems, together with the lack of industry standards and government regulations, have not significantly improved physicians access to patient medical data. For instance, some health associations, or groups, have developed centralized patient data information systems called association centric medical data (ACMD) systems that use “in-network” associations of physicians, hospitals, and medical test services providers. All patient data, including examinations, diagnoses, test results, hospital procedures, and out-patient procedures, are recorded and centrally stored at the association's information services facility. The patient typically must sign a consent form allowing authorized access by association health professionals. Association medical professionals, including the in-network patient's physician, then may access the ACMD system to review the progress of the patients' treatment and assess the results of that treatment. All data concerning the patient is managed by the association medical and IT professionals.

While these conventional ACMD systems may provide some usefulness in closed medical associations, rapidly changing healthcare remuneration systems and the lack of industry information systems standards for data interchange make access to patient data difficult if not impossible and hinder the widespread adoption of these systems. For instance, once a patient is outside the ACMD system, the patient's medical data is no longer available to the patient, physicians, or other medical health professionals. The inaccessibility of patient data is especially problematic because physician access to medical information is critical to the examination, diagnosis, and treatment of patients. Accordingly, patients and physicians need a system where patient medical data is readily and securely available.

### Patent Centric Medical Data (PCMD)

As noted above, methods, apparatuses, systems, and computer program products are described herein that provide for managing medical data using a server-based patient centric medical data (PCMD) system. The PCMD system described herein provides for improved management of medical data from multiple sources by centralizing all medical data in a single system.

In some embodiments, the present disclosure relates to a PCMD software suite that can be deployed using hardware to manage server-based PCMD by centralizing medical data, health information, and other patient data on a secure, cloud-based server or some other server hardware and software implementation. In some instances, the server may be a central repository for storing medical data centric to the patient. The server may be connected to the Internet through a wired, wireless, or hybrid communications network. Thus, the patient's data can be securely accessed anywhere in the world by a physician or patient where Internet access is available. By centralizing all patient data in one place, access to patient medical information is much easier than in conventional systems such as ACMD systems. Additionally, patients and medical professionals can easily provide, view, exchange, and update all relevant patient medical data.

In some embodiments, the PCMD software suite may comprise mobile applications that dramatically improve access to and management of medical data provided by patients, physicians, and other medical data sources. For example, the PCMD software suite may comprise patient PCMD applications that may be downloaded and installed on patient devices such as the patients' smartphones, tablet computers, or laptop computers. In another example, the PCMD software suite may comprise physician PCMD applications that may be downloaded and installed on physician devices such as the physicians' smartphones, tablet computers, or laptop computers. In yet another example, the PCMD software suite may comprise multiple patient PCMD applications and multiple physician PCMD applications that may be downloaded installed on patients' and physicians' devices to enable secure access to all patient data. Using these mobile applications, the patient and physician use their smartphones to view, edit, and update patient data. For example, the patient or physician may use a smartphone based application, or similar viewing device, that is used to view, add, or edit the patient's medical information. In all cases the patient grants access to all medical professionals and thus controls access to who can view the data.

In some embodiments, the patient may use a patient PCMD application on a patient device to add, edit, and manage the patient's medical information. For example, the patient may use the mobile application to acquire and store his/her life medical data on the server. The information may be organized such that the patient or a medical professional can easily view the patient's medical information. Additionally, the data may be encrypted such that only the patient can decrypt the data. In some instances, the data on the server may be encrypted and only the patient, or a physician temporarily authorized by the patient, can access the data. For example, the patient may grant temporary access to medical professionals to view, add or edit additional medical data about the patient using physician PCMD applications. In another example, the patient may be responsible for access, permissions, data entry and management of his/her own medical data. Thus, the patient is always in possession of the data and can securely make the data available to any medical professional anytime and anywhere.

In some embodiments, the PCMD software suite may comprise a template module to generate and transmit templates, such as patient life medical templates (PLMTs) and patient physician medical template (PPMTs), for use in organizing and storing medical information. The PLMT may be configured to capture, for example, the patient's historical medical records and other suitable data and data fields. A completed PLMT that has been saved on the patient's smartphone becomes “patient life medical record (PLMR).” The PPMT may be configured to capture, for example, physician office visit and examination information and, in some instances, may be specific to a physician's specialty. A completed PPMT that has been digitally signed (e.g., approved) by a physician becomes a “physician patient medical record (PPMR).” The PPMR may be saved to the patient's PLMR (e.g., appended thereto) and/or the physician's database. As will be recognized, templates can be stored by the PCMD system/server and can be provided to patient devices and/or physician devices.

In some embodiments, the template module may enable a patient to use a PLMT to view, add, or edit any patient medical information. In some embodiments, the template module may enable the physician or the patient to use a PPMT to record an illness complaint and illness history. In some embodiments, the physician or the patient may use a generic PPMT to record historical medical information and, in some instances, physician examinations by physicians that have not registered for access to the PCMD software suite. In some instances, the PLMT, PPMT, or both may be downloaded to the patient's device (or the physician's device) from a server in communication with the template module.

In some embodiments, the template module may enable a physician or medical professional to use the PPMT to record all examination procedures and codes, observations, diagnosis, treatments, prescriptions, and test orders. For example, the physician may use the PPMT and PLMR to evaluate a patient's complaint and previous medical history before or during a medical examination and to record diagnoses, prescriptions, medical test orders, and subsequent appointments thereafter. In some instances, the PLMR, PPMT, or both may be downloaded to the physician's device from a server in communication with the template module (or the PLMR can be provided by the patient's device). In some embodiments, the records module may store the PPMR in combination with the patient's PLMR in the patient's server database to create a permanent record of the physician visit for the patient. In some embodiments, the records module also may store the PPMR in the physician's server database for future reference. By providing templates such as PLMTs and PPMTs, the PCMD software suite allows physician examinations to be recorded and saved in patients' server databases. The PCMD software suite also allows physicians to save generic and specific patient information associated with the examination in patients' server databases, physicians' server databases, or both.

In some embodiments, the PCMD software suite may also comprise a speech-to-text (STT) module to obtain a sequence of words spoken by a user (e.g., a patient, a physician, a medical professional) and translate the spoken sequence of words into text. The template module may obtain the text from STT module. For example, the template module may access the STT module to handle speech to text dictation such that the physician and verbally dictate the results of an examination and the text is placed in the PPMT. In some embodiments, the PCMD software suite may also comprise a natural language processing (NLP) module to facilitate patient or physician text dictation, translation, transliteration, and navigation through the PLMTs, PLMRs, PPMTs, and PPMRs using text to speech dictation and speech navigation. This feature may be especially helpful for individuals who may be physically handicapped and in need of assistance filling out a PLMT and PPMT. In some embodiments, the PCMD software suite may also comprise a text-to-speech (TTS) module to obtain and vocalize text-based patient data and templates. In some instances, the functionality of the TTS module may be comprised by the STT module as a feature thereof.

In some embodiments, the PCMD software suite may also comprise an accounting module to generate accounting information, such as billing codes, and transmit the generated accounting information to a computing device. For example, the PCMD software suite may generate billing codes using a template (e.g., a PLMT, a PPMT) and/or a record (e.g., a PLMR, a PPMR) and email the generated billing codes to physician accounting personnel. These billing codes may be used for correct patient and insurance billing. In other scenarios the accounting module communicate using API to a billing system automated interface.

In some embodiments, the PCMD software suite may also comprise an appointment module to generate appointment information, such as appointment schedules and appointment reminders, and transmit the generated appointment information to a computing device. For example, the PCMD software suite may generate appointments, appointment schedules, and appointment reminders using a template (e.g., a PLMT, a PPMT) and/or a record (e.g., a PLMR, a PPMR) and email the generated information to the patient and physician office personnel.

In some embodiments, the PCMD software suite may also comprise a medical test module to generate medical test information, such as orders for medical tests, and transmit the generated medical test information to a computing device. For example, the PCMD software suite may generate orders for medical tests using a template (e.g., a PLMT, a PPMT) and/or a record (e.g., a PLMR, a PPMR) and email the generated orders to a medical test facility. The PCMD software suite may also communicate directly with a test facility's automated test management system through the use of APIs.

In some embodiments, the PCMD software suite may also comprise a pharmaceutical module to generate pharmaceutical information, such as prescriptions, and transmit the generated pharmaceutical information to a computing device. For example, the PCMD software suite may generate prescriptions using a template (e.g., a PLMT, a PPMT) and/or a record (e.g., a PLMR, a PPMR) and email the generated prescriptions to the patient's pharmacy. The PCMD software suite may also communicate directly with a pharmacy's automated prescription system through the use of APIs.

In some embodiments, the PCMD software suite may also comprise a user interface module to provide a mobile viewing application, which may be referred to herein as “ViewDock,” and/or similar words interchangeably. The ViewDock application allows patients' and physicians' to easily view PLMT and PPMT data and input data in response thereto (e.g., PLMRs, PPMRs). For instance, each PLMT and PPMT may have an electronic index. The physician may use the ViewDock application to select which section of the electronic index to view. In response to the physician's selection, the view may be displayed on the physician's device as an electronic book. The patient or physician may interact with this electronic book and easily go from page to page of the electronic book by just swiping his/her finger across the screen of his/her device.

In some embodiments, the PCMD software suite may comprise a communications security module to secure the data received, processed, stored, and transmitted by the PCMD software suite. For example, the communications security module may secure data by providing a secure credential cipher block (SCCB) application that provides for secure storage of database security credentials with no open encryption keys. In another example, the communications security module may secure data by providing a secure temporal access cipher block (STACB) application that provides for secure storage of temporal security credentials with no open encryption keys. In some instances, the STACB provides the capability for a patient to temporarily allow a physician to access the patient's medical data without exposing the patient's security encryption keys and/or other security credentials.

In some embodiments, one or more of these applications or modules may be hosted locally by a physician device or a patient device. For example, the patient PCMD application, the physician PCMD application, the template module, the records module, the STT module, the NLP module, the TTS module, the accounting module, the appointment module, the medical test module, the pharmaceutical module, the user interface module, the communications security module, any other application or module, or any combination thereof may be hosted locally by a physician device or a patient device that has been provided with specialized computerized components. In some embodiments, one or more of these applications or modules may be hosted remotely (e.g., by one or more separate devices or one or more cloud servers) and need not reside on the physician device or patient device. Thus, some or all of the functionality described herein may be provided by a third party. For example, when remotely provisioning a patient device, the patient device may access one or more third party modules via a digitizer and a telephone module, a Wi-Fi module, a software phone module, or any sort of networked connection that facilitates transmission of digitized speech to the STT module. In turn, the STT module may be connected to the other modules, such as the NLP module and the template module, to navigate and add or edit data to PLMTs and PPMTs.

In another embodiment, the PCMD mobile application may include instantaneous capture of biological information from wearable devices. For example, wearable devices can capture key information such as heart rate, blood pressure, blood-oxygen, blood-sugar, EKG, and/or other biological information. The key information may be stored in the PLMR and be available for viewing and analysis by a physician. The PCMD server could also perform real time analysis of the biological information and alert the patient of the presence of a current or impending event.

Patient centric medical data is a dramatic change with how patient medical data is managed. All of the patient's medical data is instantly and securely made available through a mobile application, and the patient can provide access to his/her information through the use of secure temporal access key. There are many advantages of these and other embodiments described herein, such as: improving the examination, diagnosis, and treatment of patients; improving access to patient data from multiple sources; improving the security of patient data; improving the ease with which patients, physicians, and medical professionals can view relevant patient medical data; providing a medical data system suitable for widespread adoption alongside rapidly changing healthcare remuneration systems and the absence of industry information systems standards for data interchange; and providing interoperability and access of the patient's medical data across the entire medical community.

### Definitions

As used herein, the terms “data,” “content,” “information,” “electronic information,” “signal,” and similar terms may be used interchangeably to refer to data capable of being transmitted, received, and/or stored in accordance with embodiments of the present disclosure. Thus, use of any such terms should not be taken to limit the spirit or scope of embodiments of the present disclosure. Further, where a first computing device or circuitry is described herein to receive data from a second computing device or circuitry, it will be appreciated that the data may be received directly from the second computing device or circuitry or may be received indirectly via one or more intermediary computing devices or circuitries, such as, for example, one or more servers, relays, routers, network access points, base stations, hosts, and/or the like, sometimes referred to herein as a “network.” Similarly, where a first computing device or circuitry is described herein as sending data to a second computing device or circuitry, it will be appreciated that the data may be sent directly to the second computing device or circuitry or may be sent indirectly via one or more intermediary computing devices or circuitries, such as, for example, one or more servers, remote servers, cloud-based servers (e.g., cloud utilities), relays, routers, network access points, base stations, hosts, and/or the like.

The term “comprising” means including but not limited to, and should be interpreted in the manner it is typically used in the patent context. Use of broader terms such as comprises, includes, and having should be understood to provide support for narrower terms such as consisting of, consisting essentially of, and comprised substantially of

The phrases “in one embodiment,” “according to one embodiment,” and the like generally mean that the particular feature, structure, or characteristic following the phrase may be included in at least one embodiment of the present disclosure, and may be included in more than one embodiment of the present disclosure (importantly, such phrases do not necessarily refer to the same embodiment).

The word “example” is used herein to mean “serving as an example, instance, or illustration.” Any implementation described herein as “example” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

The terms “processor” and “processing circuitry” are used herein to refer to any programmable microprocessor, microcomputer or multiple processor chip or chips that can be configured by software instructions (applications) to perform a variety of functions, including the functions of the various embodiments described above. In some devices, multiple processors may be provided, such as one processor dedicated to wireless communication functions and one processor dedicated to running other applications. Software applications may be stored in the internal memory before they are accessed and loaded into the processors. The processors may include internal memory sufficient to store the application software instructions. In many devices the internal memory may be a volatile or nonvolatile memory, such as flash memory, or a mixture of both. The memory may also be located internal to another computing resource (e.g., enabling computer readable instructions to be downloaded over the Internet or another wired or wireless connection).

For the purposes of this description, a general reference to “memory” refers to memory accessible by the processors including internal memory or removable memory plugged into the device, remote memory (e.g., cloud storage), and/or memory within the processors themselves. For instance, memory may be any non-transitory computer readable medium having computer readable instructions (e.g., computer program instructions) stored thereof that are executable by a processor.

The term “computing device” is used herein to refer to any one or all of programmable logic controllers (PLCs), programmable automation controllers (PACs), industrial computers, desktop computers, personal data assistants (PDAs), laptop computers, tablet computers, smart books, palm-top computers, personal computers, smartphone, headset, smartwatch, and similar electronic devices equipped with at least a processor configured to perform the various operations described herein. Devices such as smartphones, laptop computers, tablet computers, headsets, and smartwatches are generally collectively referred to as mobile devices.

As noted above, devices may include “wearable” devices. Wearable devices may collect various information from the patient. This information may be transferred to the “computing device” via wired or wireless connections. Exemplary wearable devices may include watches or bracelets devices that capture heart rate, blood pressure, blood-oxygen, blood-sugar, and/or other body information. Other wearable devices may include devices that collect biologic information.

The term “server” is used to refer to any computing device capable of functioning as a server, such as a master exchange server, web server, mail server, document server, database server, an application server, a remote server, a cloud-based server (e.g., a cloud utility), a software as a service (SaaS) server, a SaaS cloud server, or any other type of server. A server may be a dedicated computing device or a computing device including a server module (e.g., an application which may cause the computing device to operate as a server). A server module (e.g., server application) may be a full function server module, or a light or secondary server module (e.g., light or secondary server application) that is configured to provide synchronization services among the dynamic databases on computing devices. A light server or secondary server may be a slimmed-down version of server type functionality that can be implemented on a computing device, such as a smartphone, thereby enabling it to function as an Internet server (e.g., an enterprise e-mail server) only to the extent necessary to provide the functionality described herein.

The terms “circuitry,” “application,” “app,” “module,” “software module,” “utility,” “cloud utility,” “suite,” and “software suite” (or other such terms) should be understood broadly to include hardware. In some embodiments, these terms may also include software for configuring the hardware. For example, in some embodiments, “circuitry” may include processing circuitry, memory, communications circuitry, and/or input-output circuitry. In some embodiments, other elements of the present disclosure may provide or supplement the functionality of particular circuitry, modules, utilities, or suites.

As used herein, the terms “patient data,” “medical data,” “patient medical data,” “patient information,” “medical information,” “patient medical information,” “health information,” “patient health information” and similar terms may be used interchangeably to refer to data associated with one or more patients and capable of being transmitted, received, and/or stored in accordance with embodiments of the present disclosure. Thus, use of any such terms should not be taken to limit the spirit or scope of embodiments of the present disclosure. For example, one or more of these terms may refer to data or electronic information comprising or indicative of a patient's name, address, age, personal identification information (e.g., social security number, non-citizen identification number), account information, billing information, insurance information, appointments, appointment schedules, medical test orders, medical test results, examinations, diagnoses, treatments, evaluations, physician notes, patient medical history, family medical history, X-rays, computerized tomography (CT or CAT) scans, electrocardiograms (ECGs or EKGs), biopsies, blood tests, bodily fluid tests, in-patient (e.g., hospital) procedures, out-patient (e.g., medical center, clinic, physician's office) procedures, any other suitable data or information (the terms data and information are used herein interchangeably), or any combination thereof.

The terms “patient centric medical data (PCMD)” and “patient centric medical health information (PCMHI)” are used herein to refer to any concept where a patient has possession of and manages his or her own personal medical information. Normally, most patient medical data is scattered among several physician groups, hospital, and medical testing facilities. Access to this medical information by the patient or a physician is difficult if not impossible. PCMD empowers the patient with the ability to manage his/her own medical data and enable secure access to medical professionals. With the help of a PCMD smartphone application, the patient can securely store, manage, and allow access to his/her personal medical information on his/her personal phone.

The terms “PCMD application” and “PCMD app” are used herein to refer to a mobile application that supports PCMD management. The PCMD application may be transmitted to and downloaded on a patient device, a patient device, or any other suitable computing device. In some embodiments, a patient PCMD application may enable a patient to securely access and manage his/her medical data, including, but not limited to, his/her PLMR and PPMRs. In some embodiments, a physician PCMD application may allow a physician to securely access and/or edit the patient's PPMR and PLMR. The various PCMD applications described herein are platform extensible (e.g., extensible to a multitude of platforms) and can run on any mobile device or personal computer (PC) platform. Mobile device platforms include smartphone, tablet, and other mobile operating systems. PC platforms include desktop, laptop, tablet and any other PC operating systems.

The term “template” is used herein to refer to an electronic form that aids in the content and organization of information. Templates may be stored by the PCMD system in a database server and are initially blank. A template becomes a record once the template has been filled out with physician or patient information and saved to a database server or cloud account associated with the physician or patient.

The term “patient life medical template (PLMT)” is used herein to refer to a template used to create a record of the patient's life medical information and includes doctors' visits, medical procedures, and test results. In some embodiments, a patient may acquire a PLMT for the first time when the patient downloads the PCMD application and begins to enter data to the PLMT. The patient may start and stop entering data to the PLMT several times until complete. In some instances, when the patient saves the partially or fully completed PLMT, that PLMT automatically becomes a PLMR. The PLMR may comprise current medical history, family history, past medical procedures, historical medical records, and physician office visit records in the form of a PPMR. The patient may also attach historical records, images, audio, video, and other data to the PLMR. For example, the patient may upload, scan, or photograph a historical record, enter some metadata about the record, and then save that historical record to the PLMR.

The term “physician patient medical template (PPMT)” is used herein to refer to a template of the patient's complaint and history as well as the physician's examination and diagnosis. A PPMT may be unique for each medical specialty. For example, the PCMD system may comprise over one hundred unique PPMTs to cover all known medical specialties. In some embodiments, the patient may initially download a PPMT before a physician office visit and fill out the “Complaint” and “Illness History” data fields of the PPMT. During the examination, the physician may access and review the patient's PPMT complaint and illness history and proceed with the patient assessment. The physician may then record observations, tests, diagnoses, and treatments in the PPMT. Once completed, the PCMD system saves the partially or fully completed PPMT as a patient PLMR and also saves a copy of the partially or fully completed PPMR in the physician's database server (e.g., cloud server account).

The term “record” is used herein to refer to an electronic record comprising a completed template filled out with information responsive to a template. The PCMD system may generate a record by saving, to a database server, a template that has been filled out with physician or patient information.

The term “patient life medical record (PLMR)” is used herein to refer to a completed PLMT that has been saved on the patient's smartphone and/or elsewhere.

The term “physician patient medical record (PPMR)” is used herein to refer to a completed PPMT that has been digitally signed by a physician. The PPMR may be saved to the patient's PLMR and/or the physician's database server.

The term “ShareLink” is used herein to refer to a mobile WiFi Peer-to-Peer (P2P) communications technology that enables mobile to mobile device communications without the need for an Internet connection. In some embodiments, ShareLink implements at least three different connection schemes: a WiFi infrastructure connection scheme that utilizes the connection and encryptions facilities of a local WiFi router as an access point to create the P2P connection; a WiFi ad-hoc connection scheme that utilizes direct mobile phone to phone WiFi connection wherein many of the P2P connection and encryptions facilities are performed programmatically in each mobile device; and a Bluetooth connection scheme wherein, if WiFi is not available, both mobile devices may revert to Bluetooth P2P connectivity. Typically, the WiFi infrastructure connection scheme may be the fastest of these three connection schemes.

The term “ViewDock” is used herein to refer to a mobile user interface that allows a physician or patient to securely exchange data. In one illustrative example, the patient may pair his/her phone with the physician. The patient may then select the portions of his/her medical data that may be viewed by the physician. In some embodiments, ViewDock may provide a convenient index for selecting data of interest and then allowing easy page flipping to examine the specific content.

Having set forth a series of definitions called-upon throughout this application, an example system architecture is described below for implementing example embodiments and features of the present disclosure.

### System Architecture

Methods, systems, apparatuses, and computer program products of the present disclosure may be embodied by any of a variety of devices. For example, the method, system, apparatus, and computer program product of an example embodiment may be embodied by a networked device, such as one or more servers, remote servers, cloud-based servers (e.g., cloud utilities), or other network entities, configured to communicate with one or more devices, such as one or more physician devices, patient devices, or a combination thereof. Example embodiments of the physician devices and patient devices include any of a variety of stationary or mobile computing devices, such as a smartphone, mobile telephone, tablet computer, portable digital assistant (PDA), laptop computer, a desktop computer, an electronic workstation, a kiosk, or any combination of the aforementioned devices.

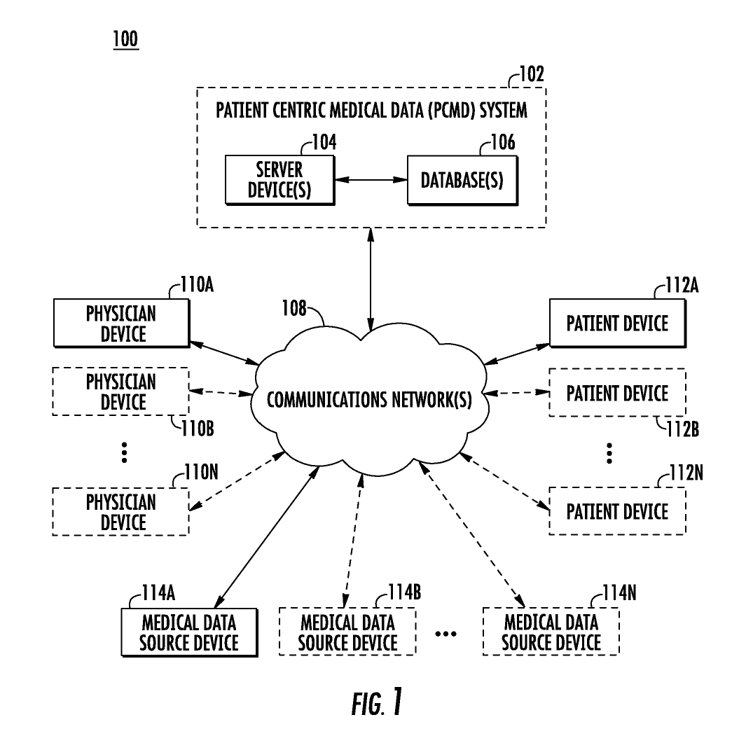

FIG. 1 illustrates a system diagram of a set of devices that may be involved in some example embodiments described herein. In this regard, FIG. 1 discloses an example computing system 100 within which embodiments of the present disclosure may operate. As illustrated, a patient centric medical data (PCMD) system 102 may be connected to one or more server devices 104 in communication with one or more databases 106. The PCMD system 102 may be connected to one or more physician devices 110A-110N, one or more patient devices 112A-112N, and one or more medical data source devices through one or more communications networks 108. In some embodiments, the PCMD system 102 may be configured to manage data, medical data, patient centric medical data (PCMD), information, health information, patient centric medical health information (PCMHI), or a combination thereof provided by one or more users (e.g., physicians, nurses, medical staff) of the one or more physician devices 110A-110N, one or more users (e.g., patients, parents, guardians, caregivers) of the one or more patient devices 112A-112N, one or more of the one or more medical data source devices 114A-114N, or a combination thereof as described in further detail below.

The PCMD system 102 may be embodied as a computer or computers as known in the art. The PCMD system 102 may provide for transmitting templates to various computing devices, including but not necessarily limited to the one or more physician devices 110A-110N, the one or more patient devices 112A-112N, or both. The PCMD system 102 may provide for receiving, from various sources, patient data provided by a user or computing device in response to a template, including but not necessarily limited to the one or more physician devices 110A-110N, the one or more patient devices 112A-112N, the one or more medical data source devices 114A-114N, or a combination thereof. In some embodiments, the PCMD system 102 may provide for generating records based on templates and patient data, such as the templates transmitted by the PCMD system 102 and the patient data received by the PCMD system 102 in response to the templates. In some embodiments, the PCMD system 102 may comprise one or more PCMD application programming interfaces (APIs) that provide communications to and from the PCMD system 102, the one or more server devices 104, the one or more databases 106, or any combination thereof. In some embodiments, the PCMD system 102 may provide for storing the generated records in various devices, such as the one or more server devices 104, the one or more databases 106, or a combination thereof. In some embodiments, the PCMD system 102 may provide mobile viewing applications (e.g., ViewDock) for viewing templates and providing patient data in response thereto. In some embodiments, the PCMD system 102 may provide communications security, such as ShareLink connections, secure credential cipher block (SCCB) applications, and secure temporal access cipher block (STACB) applications, for securing electronic communications among the various devices described herein.

The one or more server devices 104 may be embodied as one or more servers, remote servers, cloud-based servers (e.g., cloud utilities), software as a service (SaaS) servers, SaaS cloud servers, processors, or any other suitable server devices, or any combination thereof. The one or more server devices 104 receive, decrypt, process, generate, encrypt, and transmit data, signals, cryptographic information, and other electronic information to facilitate the operations of the PCMD system 102.

The one or more databases 106 may be embodied as one or more data storage devices such as a Network Attached Storage (NAS) device or devices, or as a separate database server or servers. The one or more databases 106 include information accessed and stored by the PCMD system 102 to facilitate the operations of the PCMD system 102. In some embodiments, the one or more databases 106 may store user account credentials for users of physician devices 110A-110N and patient devices 112A-112N, and/or data regarding device characteristics of various physician devices 110A-110N and patient devices 112A-112N. For example, the one or more databases 106 may comprise one or more patient databases, physician databases, template databases, password vaults, or a combination thereof for receiving, storing, and providing access to PCMD associated with users of physician devices 110A-110N, patient devices 112A-112N, medical data source devices 114A-114N, or a combination thereof. In some embodiments, the one or more databases 106 may store one or more passwords (e.g., patient passwords, physician passwords, database passwords, patient database passwords, physician database passwords), cryptographic keys (e.g., public keys, private keys, physician keys, patient keys, database keys, access keys, nonce keys, temporal keys), tokens, credentials (e.g., patient credentials, physician credentials, database credentials), index-value pairs, or a combination thereof associated with users of physician devices 110A-110N, patient devices 112A-112N, and medical data source devices 114A-114N.

The one or more physician devices 110A-110N may be embodied by any computing device known in the art. In some embodiments, the physician devices 110A-110N may comprise or be coupled to one or more smartphones, tablet computers, laptop computers, netbooks, wearable devices desktop computers, electronic workstations, kiosks, or the like. Information received by the PCMD system 102 from the one or more physician devices 110A-110N may be provided in various forms and via various methods. It will be understood, however, that in some embodiments, the physician devices 110A-110N need not themselves be physician devices, but may be peripheral devices or thin client devices communicatively coupled to physician devices. In some embodiments, the PCMD system 102 may utilize microphones, speakers, cameras, and/or displays contained in physician devices 110A-110N to provide voice and video communication with physician devices 110A-110N.

The one or more patient devices 112A-112N may be embodied by any computing device known in the art. Information received by the PCMD system 102 from these devices may be provided in various forms and via various methods. For example, the patient devices 112A-112N may be smartphones, tablet computers, laptop computers, netbooks, wearable devices, desktop computers, electronic workstations, kiosks, or the like, and the information may be provided through various modes of data transmission provided by these consumer devices. In some embodiments, the PCMD system 102 may utilize microphones, speakers, cameras, and/or displays contained in patient devices 112A-112N to provide voice and video communication with patient devices 112A-112N.

In embodiments where a physician device 110 or patient device 112 is a mobile device, such as a smartphone or tablet, the mobile device may execute an “app” (e.g., a thin-client application, a PCMD application, a ShareLink connection, a ViewDock application) to interact with the PCMD system 102. Such apps are typically designed to execute on mobile devices, such as tablets or smartphones. For example, an app may be provided that executes on mobile device operating systems such as Apple Inc.'s iOS, Google LLC's Android®, or Microsoft Corporation's Windows®. These platforms typically provide frameworks that allow apps to communicate with one another and with particular hardware and software components of mobile devices. For example, the mobile operating systems named above each provide frameworks for interacting with location services circuitry, wired and wireless network interfaces, user contacts, and other applications in a manner that allows for improved interactions between apps while also preserving the privacy and security of individual users. In some embodiments, a mobile operating system may also provide for improved communication interfaces for interacting with external devices (e.g., physician devices, patient devices). The mobile device operating system may provide APIs for providing communications with hardware and software modules executing outside of the app.

The one or more medical data source devices 114A-114N may be embodied as one or more servers, remote servers, cloud-based servers (e.g., cloud utilities), SaaS servers, SaaS cloud servers, processors, or any other suitable server devices, or any combination thereof. The one or more medical data source devices 114A-114N (which may include wearable devices) receive, decrypt, process, generate, encrypt, and transmit data, signals, cryptographic information, and other electronic information to facilitate the operations of the PCMD system 102. In some embodiments, the one or more medical data source devices 114A-114N may be associated with one or more hospitals, clinics, offices, primary physicians, specialized physicians, pharmacies, medical test facilities, clinical laboratories, insurance companies, any other suitable medical data source, or any combination thereof. In some embodiments, the one or more medical data source devices 114A-114N may comprise medical data for one or more patients. For example, the one or more medical data source devices 114A-114N may receive, store, and provide access to medical data such as patient medical history, family medical history, X-rays, computerized tomography (CT or CAT) scans, electrocardiograms (ECGs or EKGs), biopsies, blood tests, bodily fluid tests, any other suitable medical data, or any combination thereof for one or more patients (e.g., users of patient devices 112A-112N), his/her physicians and medical providers (e.g., users of physician devices 110A-110N), or both.

Additionally or alternatively, the physician devices 110A-110N, the patient devices 112A-112N, the one or more medical data source devices 114A-114N, or any combination thereof may interact with the PCMD system 102 over one or more communications networks 108. As yet another example, the physician devices 110A-110N and/or the patient devices 112A-112N may include various hardware or firmware designed to interface with the PCMD system 102. For example, the physician device 110A may be a physician device modified to communicate with the PCMD system 102, such as through the use of a physician PCMD application installed on the physician device 110A. In another example, the physician device 110B may be a purpose-built device, such as an electronic medical device offered for the primary purpose of facilitating communication between a physician device and a patient device via the PCMD system 102. In yet another example, the physician device 110B may be a purpose-built device such as a medical chatbot offered for the primary purpose of communicating with the PCMD system 102.

### Example Implementing Apparatus

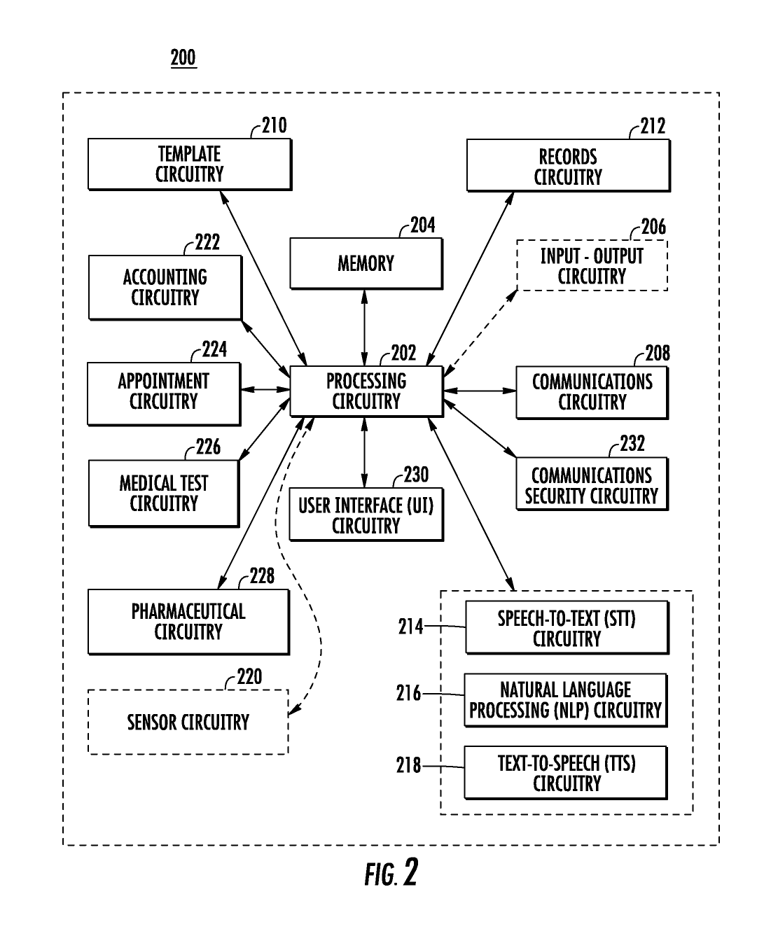

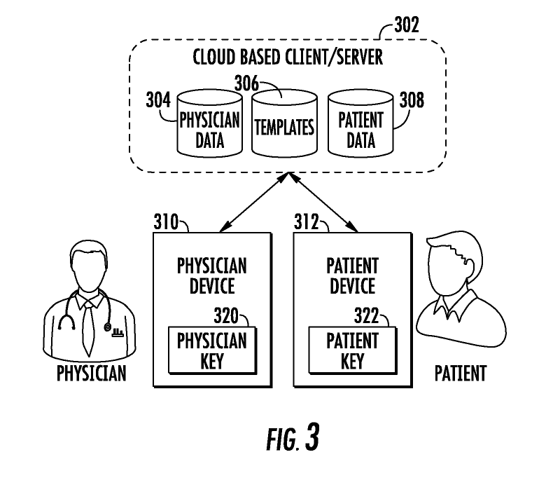

The PCMD system 102 described with reference to FIG. 1 may be embodied by one or more computing systems, such as apparatus 200 shown in FIG. 2. As illustrated in FIG. 2, the apparatus 200 may include processing circuitry 202, memory 204, input-output circuitry 206, communications circuitry 208, template circuitry 210, records circuitry 212, speech-to-text (STT) circuitry 214, natural language processing (NLP) circuitry 216, text-to-speech (TTS) circuitry 218, sensor circuitry 220, accounting circuitry 222, appointment circuitry 224, medical test circuitry 226, pharmaceutical circuitry 228, user interface (UI) circuitry 230, wearable sensor circuitry, and communications security circuitry 232. The apparatus 200 may be configured to execute the operations described above with respect to the PCMD software suite and FIG. 1 and below with respect to FIGS. 3-12.

Although some of these components 202-232 are described with respect to their functional capabilities, it should be understood that the particular implementations necessarily include the use of particular hardware to implement such functional capabilities. It should also be understood that certain of these components 202-232 may include similar or common hardware. For example, two sets of circuitry may both leverage use of the same processor, network interface, storage medium, or the like to perform their associated functions, such that duplicate hardware is not required for each set of circuitry. In some embodiments, apparatus 200 may be partially or wholly embodied as a PCMD software suite or application executing on a server device, computing device, or both. For example, apparatus 200 may be partially or wholly embodied as a PCMD software suite executing on a server device. In another example, apparatus 200 may be partially or wholly embodied as a patient PCMD application executing on a patient device. In yet another example, apparatus 200 may be partially or wholly embodied as a physician device executing on a physician PCMD application. It should also be appreciated that, in some embodiments, one or more of these components 202-232 may include a separate processor, specially configured field programmable gate array (FPGA), application specific interface circuit (ASIC), or cloud utility to perform the functions described herein.

The use of the term “circuitry” as used herein with respect to components of the apparatus 200 therefore includes particular hardware configured to perform the functions associated with respective circuitry described herein. Of course, while the term “circuitry” should be understood broadly to include hardware, in some embodiments, circuitry may also include software for configuring the hardware. For example, in some embodiments, “circuitry” may include processing circuitry, storage media, network interfaces, input-output devices, and other components. In some embodiments, other elements of the apparatus 200 may provide or supplement the functionality of particular circuitry. For example, the processing circuitry 202 may provide processing functionality, memory 204 may provide storage functionality, and communications circuitry 208 may provide network interface functionality, among other features.

In some embodiments, the processing circuitry 202 (and/or co-processor or any other processing circuitry assisting or otherwise associated with the processor) may be in communication with the memory 204 via a bus for passing information among components of the apparatus. The memory 204 may be non-transitory and may include, for example, one or more volatile and/or non-volatile memories. For example, the memory may be an electronic storage device such as a computer readable storage medium. The memory 204 may be configured to store information, data, content, applications, instructions, or the like, for enabling the apparatus to carry out various functions in accordance with example embodiments of the present disclosure. For example, the memory 204 may be configured to store patient data, templates (e.g., PLMTs, PPMTs), records (e.g., PLMRs, PPMRs), cryptographic keys, and updates thereof.

The processing circuitry 202 may be embodied in a number of different ways and may, for example, include one or more processing devices configured to perform independently. Additionally or alternatively, the processing circuitry 202 may include one or more processors configured in tandem via a bus to enable independent execution of instructions, pipelining, multithreading, or a combination thereof. The use of the term “processing circuitry” may be understood to include a single core processor, a multi-core processor, multiple processors internal to the apparatus, remote or “cloud” processors, any other suitable processor or processors, or a combination thereof.

In an example embodiment, the processing circuitry 202 may be configured to execute instructions stored in the memory 204 or otherwise accessible to the processor. Alternatively or additionally, the processor may be configured to execute hard-coded functionality. As such, whether configured by hardware or software methods, or by a combination of hardware with software, the processor may represent an entity (e.g., physically embodied in circuitry) capable of performing operations according to an embodiment of the present disclosure while configured accordingly. As another example, when the processor is embodied as an executor of software instructions, the instructions may specifically configure the processor to perform the algorithms and/or operations described herein when the instructions are executed.

In some embodiments, the apparatus 200 may include input-output circuitry 206 that may, in turn, be in communication with processing circuitry 202 to provide output to the user and, in some embodiments, to receive an indication of a user input such as a command provided by a user. The input-output circuitry 206 may comprise a user interface and may include a display that may include a web user interface (e.g., ViewDock), a mobile application, a client device, or any other suitable hardware or software. In some embodiments, the input-output circuitry 206 may also include a keyboard, a mouse, a joystick, a touch screen, touch areas, soft keys, a microphone, a speaker, or other input-output mechanisms. The processing circuitry 202 and/or input-output circuitry 206 (which may utilize the processing circuitry 202) may be configured to control one or more functions of one or more user interface elements through computer program instructions (e.g., software, firmware) stored on a memory (e.g., memory 204).

The communications circuitry 208 may be any device or circuitry embodied in either hardware or a combination of hardware and software that is configured to receive and/or transmit data from or to a network and/or any other device, circuitry, or module in communication with the apparatus 200. In this regard, the communications circuitry 208 may include, for example, a network interface for enabling communications with a wired or wireless communication network. For example, the communications circuitry 208 may include one or more network interface cards, antennae, buses, switches, routers, modems, and supporting hardware and/or software, or any other device suitable for enabling communications via a network. In some embodiments, the communication interface may include the circuitry for interacting with the antenna(s) to cause transmission of signals via the antenna(s) or to handle receipt of signals received via the antenna(s). These signals may be transmitted by the apparatus 200 using any of a number of wireless personal area network (PAN) technologies, such as Bluetooth® v1.0 through v3.0, Bluetooth Low Energy (BLE), infrared wireless (e.g., IrDA), ultra-wideband (UWB), induction wireless transmission, or any other suitable technologies. In addition, it should be understood that these signals may be transmitted using Wi-Fi, Near Field Communications (NFC), Worldwide Interoperability for Microwave Access (WiMAX) or other proximity-based communications protocols. One or more of the components 202-232 may, for instance, utilize communications circuitry 208 to communicate with a server device (e.g., one or more server devices 104), a database (e.g., one or more databases 106), a physician device (e.g., one or more of physician devices 110A-110N), a patient device (e.g., one or more of patient devices 112A-112N), a medical data source device (e.g., one or more medical data source devices 114A-114N), processing circuitry 202, memory 204, input-output circuitry 206, communications circuitry 208, template circuitry 210, records circuitry 212, STT circuitry 214, NLP circuitry 216, TTS circuitry 218, sensor circuitry 220, accounting circuitry 222, appointment circuitry 224, medical test circuitry 226, pharmaceutical circuitry 228, UI circuitry 230, and communications security circuitry 232, or any other suitable circuitry or device.

The template circuitry 210 includes hardware components designed or configured to generate, retrieve, and transmit templates such as PLMTs and PPMTs. For example, the template circuitry 210 may utilize communications circuitry 208 to communicate with a patient device (e.g., patient device 112) and thus be configured to generate, or retrieve, and transmit one or more templates (e.g., PLMTs, PPMTs) to the patient device. In another example, the template circuitry 210 may utilize communications circuitry 208 to communicate with a physician device (e.g., physician device 110) and thus be configured to generate, or retrieve, and transmit one or more templates (e.g., PPMTs) to the physician device.

In some embodiments, the template circuitry 210 may include hardware components designed or configured to enable a patient to use a PLMT to view, add, or edit any patient medical information. In some embodiments, the template circuitry 210 may enable the patient, using a patient device, to use a PPMT to record an illness complaint and illness history. In some embodiments, the patient may use a generic PPMT to record historical medical information and, in some instances, physician examinations by physicians that have not registered for access to the PCMD system. In some instances, the PLMT, PPMT, or both may be downloaded to the patient device from a server in communication with template circuitry 210. In some embodiments, the template circuitry 210 may be in communication with the records circuitry 212 to store the PLMT, the PPMT, and patient data provided in response thereto in the patient's server database or cloud account as a record (e.g., PLMR, PPMR).

In some embodiments, the template circuitry 210 may include hardware components designed or configured to enable a physician or medical professional to use the PPMT to record all examination procedures and codes, observations, diagnosis, treatments, prescriptions, and test orders. For example, the physician, using a physician device, may use a PPMT and the patient's PLMR to determine the patient's complaint and previous medical history before or during a medical examination and to record diagnoses, prescriptions, medical test orders, and subsequent appointments thereafter. In some instances, the PLMT, PPMT, or both may be downloaded to the physician device from a server in communication with the template circuitry 210. In some embodiments, the template circuitry 210 may be in communication with the records circuitry 212 to store the PPMT in combination with the PLMT and patient data provided in response to the PPMT and the PLMT in the patient's server database or cloud account as a first record (e.g., PLMR). In some embodiments, the template circuitry 210 may be in communication with the records circuitry 212 to store the PPMT and patient data provided in response thereto in the physician's server database or cloud account as a second record (e.g., PPMR).

The records circuitry 212 includes hardware components designed or configured to receive templates, patient data, or both and generate records, such as PLMRs and PPMRs, based on the received templates, patient data, or both. For example, the records circuitry 212 may utilize communications circuitry 208 to communicate with a patient device (e.g., patient device 112) and thus be configured to receive, from the patient device, first patient data (e.g., complaints, medical history, images, audio, videos, sensor-based data such as glucose measurements and patient-performed bodily fluid tests, etc.) provided in response to a first template, such as a PLMT transmitted to the patient device by the template circuitry 210. In another example, the records circuitry 212 may further utilize communications circuitry 208 to communicate with a physician device (e.g., physician device 110) and thus be configured to receive, from the patient device or the physician device, second patient data (e.g., examination notes, medical test results, diagnoses, etc.) provided in response to a second template, such as a PPMT transmitted to the patient device or the physician device by the template circuitry 210. In some embodiments, the records circuitry 212 may generate a first record (e.g., PLMR), based on the first template (e.g., PLMT) and the first patient data. In some embodiments, the records circuitry 212 may generate a second record (e.g., PPMR), based on the second template (e.g., PPMT) and the second patient data. In some embodiments, the records circuitry 212 may store the first record (e.g., PLMR) in the patient's database server or cloud account. In some embodiments, the records circuitry 212 may store the second record (e.g., PPMR) as part of the patient's PLMR in the physician's database server or cloud account.

The STT circuitry 214 includes hardware components designed or configured to receive electronic information indicative of a sequence of words spoken by a user, generate electronic information indicative of a textual representation of the sequence of words spoken by the user based on the electronic information indicative of a sequence of words spoken by a user, and transmit the electronic information indicative of the textual representation of the sequence of words spoken by the user to the template circuitry 210, records circuitry 212, or any other circuitry or component described herein. These hardware components may, for instance, utilize communications circuitry 208 to communicate with a physician device (e.g., one or more of physician devices 110A-110N), a patient device (e.g., one or more of patient devices 112A-112N, which may include wearable devices), or any other suitable circuitry or device. For example, the STT circuitry 214 may be in communication with a patient device (e.g., patient device 112), and thus configured to receive, via communications circuitry 208, patient data in the form of electronic information indicative of a sequence of words spoken by the patient to the patient device. In another example, the STT circuitry 214 may be in communication with a physician device (e.g., physician device 110), and thus configured to receive, via communications circuitry 208, patient data in the form of electronic information indicative of a sequence of words spoken by the physician to the physician device. In yet another example, the STT circuitry 214 may be in communication with the records circuitry 212, and thus configured to transmit the electronic information indicative of the textual representation of the sequence of words spoken by the user to the records circuitry 212, via communications circuitry 208. The STT circuitry 214 may utilize processing circuitry 202, NLP circuitry 216, or both to perform the above operations, and may utilize memory 204 to store collected electronic information indicative of sequences of words spoken by users, electronic information indicative of textual representations of those sequences of words, or other electronic information.

The NLP circuitry 216 includes hardware components designed or configured to receive a textual representation of patient data, appointment reminders, medical test orders, prescriptions, sequences of words, and other data, generate a translation (e.g., a translation of a natural language, such as a translation from Spanish to English) or transliteration (e.g., a transliteration from a Romanization of Mandarin (e.g., pinyin) to Mandarin characters) of the textual representation, and transmit translation or transliteration to any of the components 202-232. In some instances, these hardware components may, for instance, utilize communications circuitry 208 to communicate with a physician device (e.g., one or more of physician devices 110A-110N), a patient device (e.g., one or more of patient devices 112A-112N), or both, and thus may be configured to transmit the translation or transliteration to the physician device, the patient device, or both via communications circuitry 208. In some instances, these hardware components may be configured to transmit the translation or transliteration to the TTS circuitry 218. The NLP circuitry 216 may utilize processing circuitry 202 to perform the above operations, and may utilize memory 204 to store collected translations, transliterations, or other data. In some embodiments, the STT circuitry 214 may partially or wholly comprise the NLP circuitry 216.

The TTS circuitry 218 includes hardware components designed or configured to receive a textual representation of patient data, appointment reminders, medical test orders, prescriptions, translations, transliterations, sequences of words, and other data, generate electronic information indicative of a vocal representation of the textual representation, and transmit the electronic information indicative of the vocal representation to the input-output circuitry 206. These hardware components may, for instance, utilize communications circuitry 208 to communicate with a physician device (e.g., one or more of physician devices 110A-110N), a patient device (e.g., one or more of patient devices 112A-112N), or both, and thus may be configured to transmit the electronic information indicative of the vocal representation to the physician device, the patient device, or both via communications circuitry 208. The TTS circuitry 218 may utilize processing circuitry 202, NLP circuitry 216, or both to perform the above operations, and may utilize memory 204 to store collected electronic information indicative of the vocal representations or other data. In some embodiments, the STT circuitry 214 may partially or wholly comprise the TTS circuitry 218.

The sensor circuitry 220 may be embodied as any device or means embodied in circuitry, hardware, a computer program product comprising computer readable program instructions stored on a computer readable medium (e.g., memory 204) and executed by a processing device (e.g., processing circuitry 202). In some embodiments, sensor circuitry 220 may include one or more sensors, such as a bodily fluid analyzer (e.g., blood analyzer, urine analyzer, glucose sensor), a cytometric sensor, a photoplethysmographic sensor, a cardiographic sensor (e.g., EKG sensor), a medical imaging sensor, an ambient light sensor, a proximity sensor, an accelerometer, a gyroscope, a magnetometer, a pressure sensor, an altimeter, a barometric pressure sensor, a temperature sensor, a photodetector, a motion sensor, any other suitable sensor, or any combination thereof, such as a microelectromechanical lab-on-a-chip. These sensors may be implemented in wearable devices like a wrist watch, electronic wrist band, or other wearable device. Sensor circuitry 220 may be configured to receive and/or transmit any data that may be stored by memory 204 using any protocol that may be used for communications between computing devices.

The accounting circuitry 222 includes hardware components designed or configured to generate accounting information, such as billing codes (e.g., insurance billing codes), and transmit the generated accounting information to a computing device. For example, the accounting circuitry 222 may utilize communications circuitry 208 to communicate with a physician device (e.g., physician device 110), a patient device (e.g., patient device 112), or both and thus be configured to generate, or retrieve, and transmit one or more billing codes to the physician device, the patient device, or both. In some embodiments, the accounting circuitry 222 may generate the accounting information based on a template, such as an accounting template, a PLMT, a PPMT, or a combination thereof.

The appointment circuitry 224 includes hardware components designed or configured to generate appointment information, such as appointment schedules and appointment reminders, and transmit the generated appointment information to a computing device. For example, the appointment circuitry 224 may utilize communications circuitry 208 to communicate with a physician device (e.g., physician device 110), a patient device (e.g., patient device 112), or both and thus be configured to generate, or retrieve, and transmit one or more appointments, appointment schedules, and appointment reminders to the physician device, the patient device, or both. In some embodiments, the appointment circuitry 224 may generate the appointment information based on a template, such as an appointment template, a calendar, a PLMT, a PPMT, or a combination thereof.

The medical test circuitry 226 includes hardware components designed or configured to generate medical test information, such as orders for medical tests (e.g., blood tests, biopsies, CAT scans, etc.), and transmit the generated medical test information to a computing device. For example, the medical test circuitry 226 may utilize communications circuitry 208 to communicate with a physician device (e.g., physician device 110), a patient device (e.g., patient device 112), or both and thus be configured to generate, or retrieve, and transmit one or more orders for medical tests to the physician device, the patient device, or both. In some embodiments, the medical test circuitry 226 may generate the medical test information based on a template, such as a medical test template, a PLMT, a PPMT, or a combination thereof.

The pharmaceutical circuitry 228 includes hardware components designed or configured to generate pharmaceutical information, such as prescriptions, and transmit the generated pharmaceutical information to a computing device. For example, the pharmaceutical circuitry 228 may utilize communications circuitry 208 to communicate with a physician device (e.g., physician device 110), a patient device (e.g., patient device 112), or both and thus be configured to generate, or retrieve, and transmit one or more prescriptions to the physician device, the patient device, or both. In some embodiments, the pharmaceutical circuitry 228 may generate the pharmaceutical information based on a template, such as a pharmaceutical template, a PLMT, a PPMT, or a combination thereof.

The UI circuitry 230 includes hardware components designed or configured to provide a mobile viewing application, such as ViewDock, for viewing PLMTs, PLMRs, PPMTs, and PPMRs and providing patient data in response thereto. For example, the UI circuitry 230 may utilize communications circuitry 208 to communicate with a physician device (e.g., physician device 110), a patient device (e.g., patient device 112), or both and thus be configured to generate and transmit ViewDock display screens to the physician device, patient device, or both, and receive patient data provided in response thereto. In another example, the UI circuitry 230 may utilize input-output circuitry 206 to generate ViewDock display screens and receive patient data input in response thereto.

The communications security circuitry 232 includes hardware components designed or configured to secure data, signals, and electronic information received, processed, stored, and transmitted by the apparatus 200. For example, the communications security circuitry 232 may utilize communications circuitry 208 to communicate with a physician device (e.g., physician device 110), a patient device (e.g., patient device 112), or both and thus be configured to encrypt and decrypt data transmitted to and received from the physician device, the patient device, or both. In some embodiments, the communications security circuitry 232 includes hardware components designed or configured to secure data by providing a ShareLink connection between computing devices, such as between a patient device and a physician device. In some embodiments, the communications security circuitry 232 includes hardware components designed or configured to secure data by providing a secure credential cipher block (SCCB) application that provides for secure storage of database security credentials with no open encryption keys. In some embodiments, the communications security circuitry 232 includes hardware components designed or configured to secure data by providing a secure temporal access cipher block (STACB) application that provides for secure storage of temporal security credentials with no open encryption keys. In some instances, the STACB provides the capability for a patient device to temporarily allow a physician device to access patient data without exposing the patient device's security encryption keys.

As will be appreciated, any such computer program instructions and/or other type of code may be loaded onto a computer, processor or other programmable apparatus's circuitry to produce a machine, such that the computer, processor other programmable circuitry that execute the code on the machine create the means for implementing various functions, including those described herein.

As described above and as will be appreciated based on this disclosure, embodiments of the present disclosure may be configured as systems, apparatuses, methods, mobile devices, backend network devices, computer program products, other suitable devices, and combinations thereof. Accordingly, embodiments may comprise various means including entirely of hardware or any combination of software and hardware. Furthermore, embodiments may take the form of a computer program product on at least one non-transitory computer-readable storage medium having computer-readable program instructions (e.g., computer software) embodied in the storage medium. Any suitable computer-readable storage medium may be utilized including non-transitory hard disks, CD-ROMs, flash memory, optical storage devices, or magnetic storage devices.

The physician devices 110A-110N, patient devices 112A-112N, and one or more medical data source devices 114A-114N may be embodied by one or more computing devices or systems that also may include processing circuitry, memory, input-output circuitry, and communications circuitry. For example, a patient device 112 may be a smartphone on which an app (e.g., a patient PCMD app) is running or otherwise being executed by processing circuitry. In another example, a physician device 110 may be a smartphone on which an app (e.g., a physician PCMD app) is running or otherwise being executed by processing circuitry. As it relates to operations described in the present disclosure, the functioning of these components may be similar to the similarly named components described above with respect to FIG. 2. Additional description of the mechanics of these components is omitted for the sake of brevity. These device components, operating together, provide the respective apparatuses with the functionality necessary to facilitate the communication of data (e.g., templates, patient data, records) with the PCMD system described herein.

FIG. 3 illustrates a system diagram of a set of devices that may be involved in providing server-based PCMD in accordance with some example embodiments described herein. In general terms, FIG. 3 highlights the relationship between the patient and physician data exchange. As shown in FIG. 3, the PCMD server 302 may be a cloud based server and comprise physician data database 304, templates database 306, and patient data database 308. The physician data database 304 may store physician data such as PPMRs and patient data provided in response to PPMTs. The templates database 306 may store templates such as PLMTs and PPMTs. The patient data database 308 may store patient data such as PLMRs, PPMRs, and patient data provided in response to PLMTs and PPMTs. The PCMD server 302 may be connected to physician device 310 and patient device 312 via a communications network.

In some embodiments, patient and physician medical data may be securely stored on the PCMD server 302, such as in patient data database 308 and physician data database 304. In some embodiments, the patient and physician data may be encrypted and may only be accessed using secure keys, such as physician key 320 and patient key 322, respectively stored on the physician device 310 and the patient device 312. In some embodiments, the physician key 320 may be generated when the physician PCMD application is initially downloaded on the physician device 310. In some embodiments, the patient key 322 may be generated when the patient PCMD application is initially downloaded on the patient device 312. When the patient wishes to share his/her medical data, the patient PCMD application executing on the patient device 312 creates a unique random security key, such as patient key 322, and transmits, either directly (e.g., via a ShareLink connection, which may only provide a read only copy of the medical data for viewing) or indirectly (e.g., via the PCMD server 302), the security key to the physician device 310. In some embodiments, the patient's security key is temporal and the length of time that it is valid is programmable by a user (e.g., using a mobile viewing application such as ViewDock; using the input-output circuitry 206 of the apparatus 200; or both). In one illustrative example, the temporal value may be set for a length of time of one hour. Additionally, the shared data may be read only in certain embodiments.

In some embodiments, patient medical data may be organized using templates (e.g., PLMTs, PPMTs) stored in templates database 306. The templates may be medical forms that facilitate organization of the patient's medical information. The templates may be downloaded from templates database 306 via the PCMD server 302. Once the templates have been filled out they may be referred to as records (e.g., PLMRs, PPMRs). The PLMR comprises patient personal medical data. PPMTs may be unique to the physician's medical specialty and used to record the patient's examination, tests, diagnosis, and treatment procedures. Once the examination is complete and the patient data is provided by the physician in response to the PPMT, a PPMR is generated and stored as a PPMR (e.g., in physician data database 304) and/or as part of the patient's PLMR (e.g., in the patient data database 308). The patient's PLMR may have one or more PPMRs over time as the patient undergoes many physician examinations and medical procedures.

In some embodiments, during initial registration, the physician may download the physician PCMD application from the PCMD server 302 and install the downloaded physician PCMD application on the physician device 310. The physician may use the physician PCMD application executing on the physician device 310 to enter practice administrative information and select a practice specific PPMT. For example, the PPMT may be unique to the physician's medical practice. The physician may store the practice account information to the PCMD server 302 in the physician's database (e.g., physician data database 304). Subsequently, when a patient makes an office appointment with the physician, the practice specific PPMT may be downloaded from the PCMD server 302 to the physician device 310.

In some embodiments, the patient initially downloads the patient PCMD application from the PCMD server 302 and installs the patient PCMD application on the patient device 312. The patient PCMD application may download a PLMT from the PCMD server 302 and request the patient to fill out the PLMT. The PLMT comprises personal information, medical history, family medical history, current medications and allergies. The patient data provided by the patient in response to the PLMT is used to generate a PLMR. The PLMR may be stored in the patient's database (e.g., patient data database 308). In some embodiments, the patient may also attach any relevant previous medical records to the PLMR. For example, the patient may select a “generic” PPMT, attach the records, and store the PPMT and attached records to the PLMR.

In some embodiments, when the patient schedules a physician office visit, a physician specific PPMT is downloaded from the PCMD server 302 to the patient device 312. The patient may fill out the medical complaint and illness history before arriving at the physician's office. At the start of the examination, the patient may pair the patient device 312 with the physician device 310. The pairing may be securely accomplished by the patient device 312 by sending a short message service (SMS) message along with a secure pairing key (e.g., patient key 322) to the physician device 310. The pairing key may provide access to the patient's data. In some embodiments, the pairing key is temporal and only gives the physician access to the patient's data for about an hour. Once expired, the physician can no longer access the patient's data. At the start of the examination the physician examines the patient's PLMR and/or PPMT and evaluates the patient's complaint. The physician then performs the examination, orders tests, and eventually creates a diagnosis and treatment. Throughout the process the physician uses the PPMT to record all observations. The physician may also attach documents, test results, pictures and downloads to the PPMT.

In some embodiments, the PPMT may be used in combination with electronic dictation and video. For example, the physician may use electronic dictation to record examination notes during the examination. The physician may select dictation mode and then speak into the physician device 310. Using PPMT navigation queues, the physician may verbally guide the physician PCMD application to the desired section of the PPMT. The physician then may dictate examination notes, and the PCMD application may convert the spoken examination notes text. The physician may also use video dictation. For example, the physician may use the phone camera and directly provide video and audio of the examination results. Video dictation provides a much more interactive multimedia presentation to the patient.

In some embodiments, once the examination is complete, the physician device 310 may transfer the PPMT and the patient data provided by the physician in response to the PPMT to generate a PLMR stored in the patient's database (e.g., patient data database 308). The device pairing may be disconnected and the physician may no longer access the patient's data. The completed examination PPMR also may be stored in the PCMD system 302 in the physician's database (e.g., physician data database 304) for future reference.

### Server-Based PCMD Architecture

In some embodiments, the server-based PCMD architecture comprises three key components: a cloud based PCMD web API server; a hybrid patient PCMD application; and a hybrid physician PCMD application. The web PCMD API server may provide the PCMD data management services and security. The patient and physician PCMD applications provide a rich user experience (UX) interface and help organize the viewing and data entry. The server-based PCMD architecture integrates these three key components together to provide a highly functional and secure interface to the patient's data and the physician's data.

### Web API Services

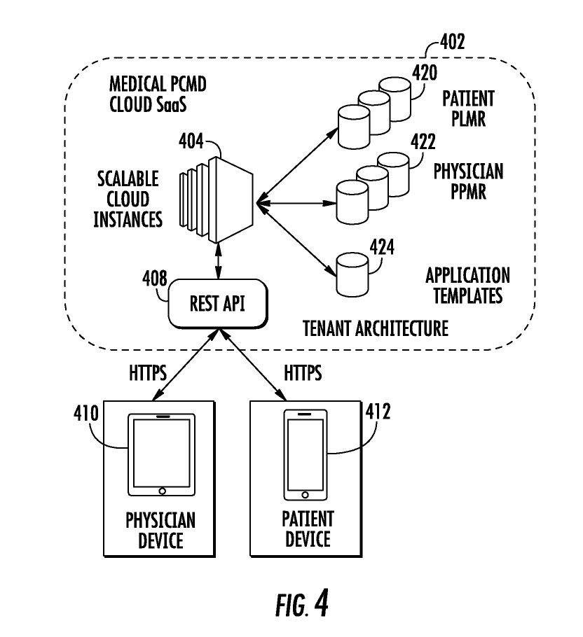

In some embodiments, the web API services comprise a set of services organized as a Software as a Service (SaaS). Accordingly, the web API is a common service which all patient devices and physician devices access as a singular resource. FIG. 4 highlights the SaaS services.

FIG. 4 illustrates a system diagram of a set of devices that may be involved in providing a PCMD SaaS cloud server 402 in accordance with some example embodiments described herein. As shown in FIG. 4, the PCMD SaaS cloud server 402 may comprise one or more scalable cloud instances 404, a representational state transfer (REST) API 408, one or more patient databases 420 (e.g., storing patient data and one or more records such as PLMRs and PPMRs), one or more physician databases 422 (e.g., storing physician data and one or more records such as PPMRs), and one or more application template databases 424 (e.g., storing one or more application templates such as PLMTs and PPMTs). The PCMD SaaS cloud server 402 may be connected to physician device 410 and patient device 412 through REST API 408 via a communications network. In some embodiments, the PCMD SaaS cloud server 402 may transmit data to and receive data from fast healthcare interoperability resources (FHIR)-based systems and devices (e.g., one or more of the medical data source devices 114 shown in FIG. 1) via REST API 408.

In some embodiments, requests for service may be made through REST API calls via REST API 408. The API Services may handle all data editing/updates, data views, data storage, and PLMR and PPMR management. In some instances, API commands may be made over a standard HTTP internet connection. HTTP typically provides excellent interoperability across multiple viewing platforms including multiple mobile device manufacturers. HTTP also enables wide geographic remote internet access to patient or physician data. In some embodiments, the hybrid patient PCMD application and the hybrid physician PCMD application make API calls to the PCMD SaaS cloud server 402. The API calls may initiate the PCMD SaaS cloud server 402 to perform various work. Units of work, or requests, may be broken down into worker threads. The worker threads may be stateless and configured to run across multiple server instances (e.g., one or more scalable cloud instances 404) all at once. As the number of patients and physician requests increases, the PCMD SaaS cloud server 402 also may increase the number of worker threads. As a server instance becomes over stressed with worker thread requests, the PCMD SaaS cloud server 402 may automatically start another server instance and move all new work to the new instance. This architecture enables scalable performance as the number of requests increase or decrease.

In some embodiments, stateless worker threads may also allow for server instance failures. For example, if a server instance fails (e.g., due to software or hardware failures), the PCMD SaaS cloud server 402 may take the instance off-line and restart the worker thread on a new server instance. As the requests for service go up or down, the PCMD SaaS cloud server 402 may scale resources to match the demand.

In some embodiments, the PCMD SaaS cloud server 402 may implement a tenant data storage architecture. Many database implementations intermix multiple patient and physician data. In contrast, the tenant data storage architecture creates a unique database for each individual patient and physician. Each database contains its own security credentials and thus keeps patient and physician data separate, more secure and less vulnerable to coding errors that may create security vulnerabilities.

### Web Security Services

In some embodiments, the web API service may incorporate a unique secure access technology called Secure Credentials Cipher Block (SCCB). SCCB is an innovative and secure way to access and protect patient and physician data. Normally, encryption at rest requires exposed encryption keys stored somewhere on the server. In contrast, SCCB is a technology that never allows open security keys on the server. The keys always remain securely stored on the patient device or the physician device. SCCB also implements a secure temporal key methodology that allows patients to share data with medical professionals without exposing the patient's keys.

### Secure Patient Medical Data Access

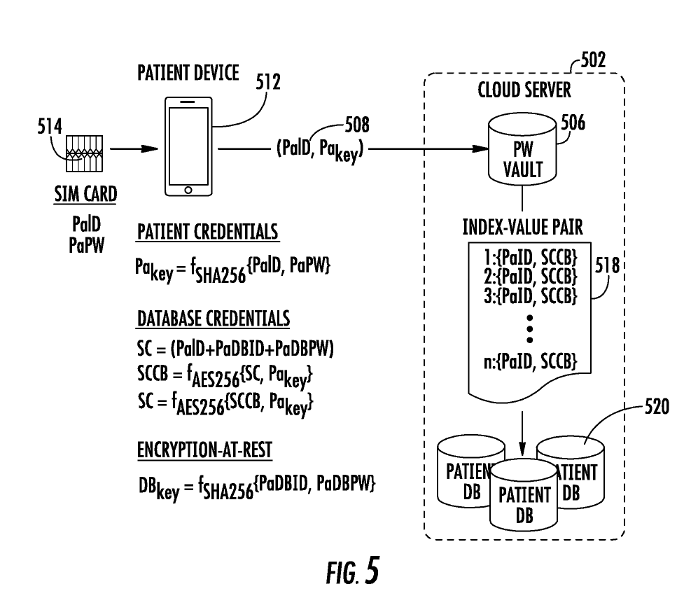

The patient's application may access its data by an API call to the PCMD web API server. The secure sequence is highlighted in FIG. 5.

FIG. 5 illustrates a schematic diagram for providing patient secure credentials in accordance with some example embodiments described herein.

As shown in FIG. 5, the PCMD server 502 may comprise a password vault 506, index-value database 518, and one or more patient databases 520. The PCMD server 502 may be an API server. In some embodiments, the patient device 512 may first negotiate with the PCMD server 502 for a set of HTTPS session keys. HTTPS uses the servers X.509 public/private key certificate to generate a secure set of session keys. The specific key exchange algorithm to be used may be negotiated at the start of the request. Typically, the key exchange algorithm comprises 2096-bit RSA asymmetric encryption. The data encryption algorithm typically comprises AES256 symmetric encryption. In many instances, HTTPS ensures the data communications between the phone and server are encrypted and secure.

In some embodiments, once the keys are exchanged, the patient PCMD application may request API services using keyed-hash message authentication code (HMAC) security. For example, the patient device 512 may transmit an HMAC 508 comprising the patient's ID (PaID) and the patient's key (Pa_{key}) to the PCMD server 502. HMAC may be used to validate that the patient device 512 has the proper security credentials to access the patient's data. The HMAC may be composed of the patient's ID (PaID) and the patient's key (Pa_{key}). In some embodiments, the Pa_{key} may be a SHA256 hash of the concatenation of the patient ID and Password (PaID+PaPW). The API server may validate the HMAC by comparing HMAC hash with the one stored in the password vault 506.

Next, the PCMD server 502 may transmit a request to the password vault 506 using the patient's ID as an index to find the SCCB. The PCMD server 502 then uses the Pa_{key} and AES256 to decrypt the SCCB. The PaID and SCCB pairs are shown in FIG. 5 as index-value database 518. The patient's ID (PaID), patient database ID (PaDBID) and patient database password (PaDBPW) then are returned. The database may validate the correct PaID. If they match, the patient database 520 may be accessed using the PaDBID and PaDBPW. The returned data may be transmitted to the patient device 512 and is decrypted using AES256 and the Pa_{key}.

In some embodiments, the patient's data encryption key may be securely created on the patient device. For example, the key, Pa_{key}, is a SHA256 hash of the PaID and PaPW. The PaID may be a known component and the PaPW may be a secret only know to the patient. In some embodiments, the key is never stored on the server and any security credentials are always encrypted with the Pa_{key}. In some instances, the data on the server can never be decrypted without the key. No one, including the physician and database and cloud server administrators, can view the patient's data except the patient. In some instances, the PaID and PAPW optionally may be stored in a subscriber identification module (SIM) card 514 of patient device 512 for easy key recovery so that the patient may recover his/her medical data if the patient forgets his/her password.

### Secure Physician Medical Data Access

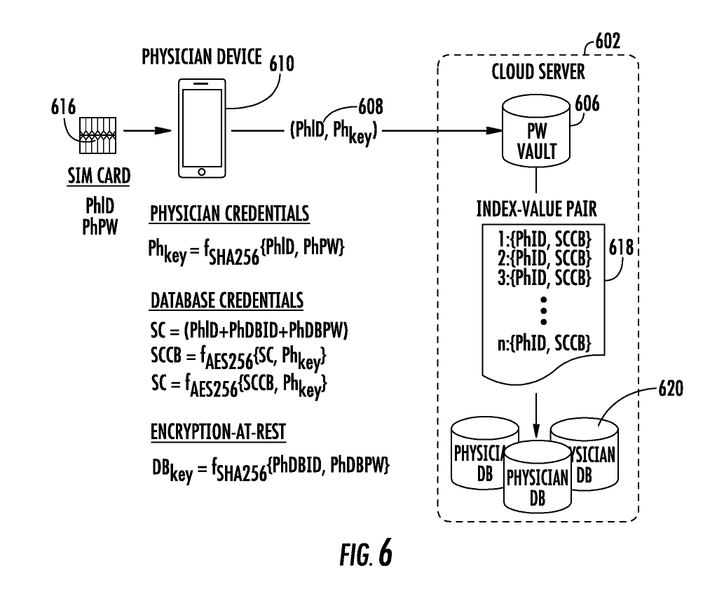

In some embodiments, physician data access may be similar to the patient data access described above with reference to FIG. 5. The security architecture for physician data access is highlighted in FIG. 6.

FIG. 6 illustrates a schematic diagram for providing physician secure credentials in accordance with some example embodiments described herein. As shown in FIG. 6, the PCMD server 602 may comprise a password vault 606, index-value database 618, and one or more physician databases 620. In some embodiments, the physician device 610 may request data from the physician database 620 by issuing an API call to the PCMD server 602. The request may be initiated by an HTTPS secure session request. The PCMD server 602 and the physician device 610 may exchange secure encryption keys and the encrypted session may be started. The physician PCMD application executing on the physician device 610 may transmit the secure request using an HMAC 608 passing the physicians ID (PhID) and a SHA256 hash of the physician's PhID and password (PhPW) or (PhID+PhPW). A request to the database may be initiated by using the PhID to find the SCCB in the database. The SCCB may be decrypted using the hash and returning the Physician ID (PhID), database ID (PhDBID) and PW (PhDBPW). The physician database 620 may be access by using the PhDBID and PhDBPW. The returned data may be encrypted and returned to the physician device 610 and decrypted using the Phkey. The PhID and PhPW optionally may be stored in a SIM card 616 of the physician device 610.

### Secure Physician Patient Medical Data Access

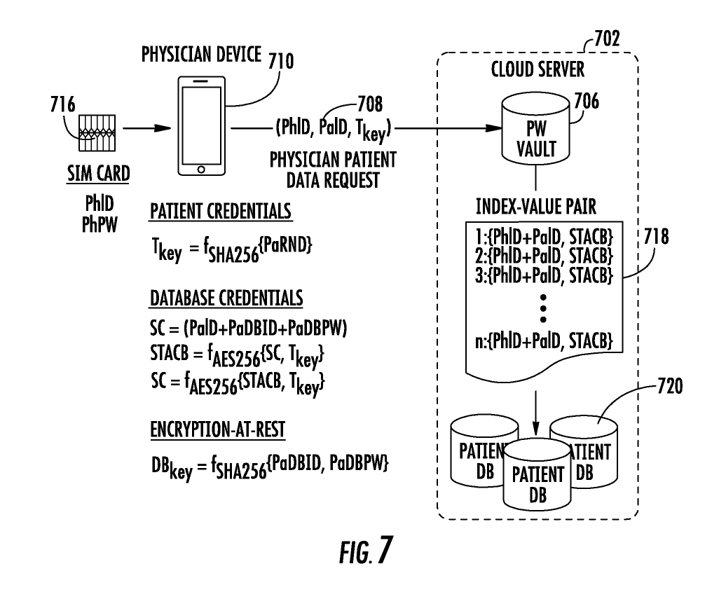

In some embodiments, secure physician access of the patient's medical data may be more complex than secure patient access of the patient's own medical data. The patient's security credentials typically may be encrypted using the Pa_{key}, which must always remain secret. One highly secure method for physician access is the use of a temporal key to access the patient's database credentials for accessing the patient's data. The temporal key may be generated by the patient device. The temporal key may have the ability to access the patient's data only for a short period of time. Once the temporal key has expired, the temporal key can no longer access the patient's data. The secure credentials are referred to herein as Secure Temporal Access Cipher Block (STACB). In some embodiments, the STACB uses the same password vault management scheme as the SCCB. The patient/physician temporal key architecture is highlighted in FIG. 7.

FIG. 7 illustrates a schematic diagram for providing patient and physician secure access in accordance with some example embodiments described herein. As shown in FIG. 7, the PCMD server 702 may comprise a password vault 706, index-value database 718, and one or more patient databases 720. In some embodiments, at the start of an examination, the patient may virtually pair his/her patient device with the physician's physician device. For example, the patient device first may generate a temporal key and STACB. The patient device also may generate a random number and creates a hash using SHA256 to be the temporal key (T_{key}).

Next, the patient device may create a STACB by transmitting an API request to the PCMD server 702. The API datagram may be encrypted using HTTPS and contain the patient's PaID, Pa_{key} and T_{key}. The PCMD server 702 may use the PaID and Pa_{key} to retrieve the patient's SCCB. The PCMD server 702 may decrypt the SCCB and return the PaID, DBID, and DBPW. The PaID first may be validated. If valid, a STACB JSON data structure may be created using the PaID, PaDBID, PaDBPW, Pa_{key}, and a time stamp (Ts). In some embodiments, the time stamp may be the current time in coordinate d universal time (UTC). The STACB may be encrypted using the T_{key} and placed in the password vault 706 using the data structure pointer concatenation of {PaID+PhID}. The data structure pointer may be unique to the patient and physician. The patient device then may transmit a secure SMS message to the physician device 710 that comprises the temporal key T_{key}. The physician device 710 may store the temporal key and use it to access the patient's data stored in patient database 720. In some embodiments, the PhID and PhPW optionally may be stored in a SIM card 716 of the physician device 710.

The physician device 710 may retrieve patient data with an HTTPS API request 708 to the PCMD server 702. The API datagram may contain the PhID, PaID, Ph_{key} and T_{key}. The PCMD server 702 may create the STACB pointer using the concatenation of the PaID and PhID. The STACB may be retrieved and then decrypted using the T_{key}. The PaID, PaDBID, PADBPW, Pa_{key}, and Ts may be extracted from the JSON data structure. The PCMD server 702 first may verify the correct PaID. If the PaID is correct, the PCMD server 702 then may verify that the token has not expired. Subsequently, the current UTC time (Tc) may be subtracted from Ts. If the difference exceeds a preset time (Tp), the token may be considered expired and thus invalid. Usually, Tp may be set for a length of time of one hour.

If the token is valid, the PCMD server 702 may extract and encrypt the patient's data using the Ph_{key} and return the patient's data to the physician device 710. If the token is invalid, the token may be removed from the password vault 706 and a token expired error message may be generated and returned to the physician device 710. As a result, the token is no longer available to access the patient's data.

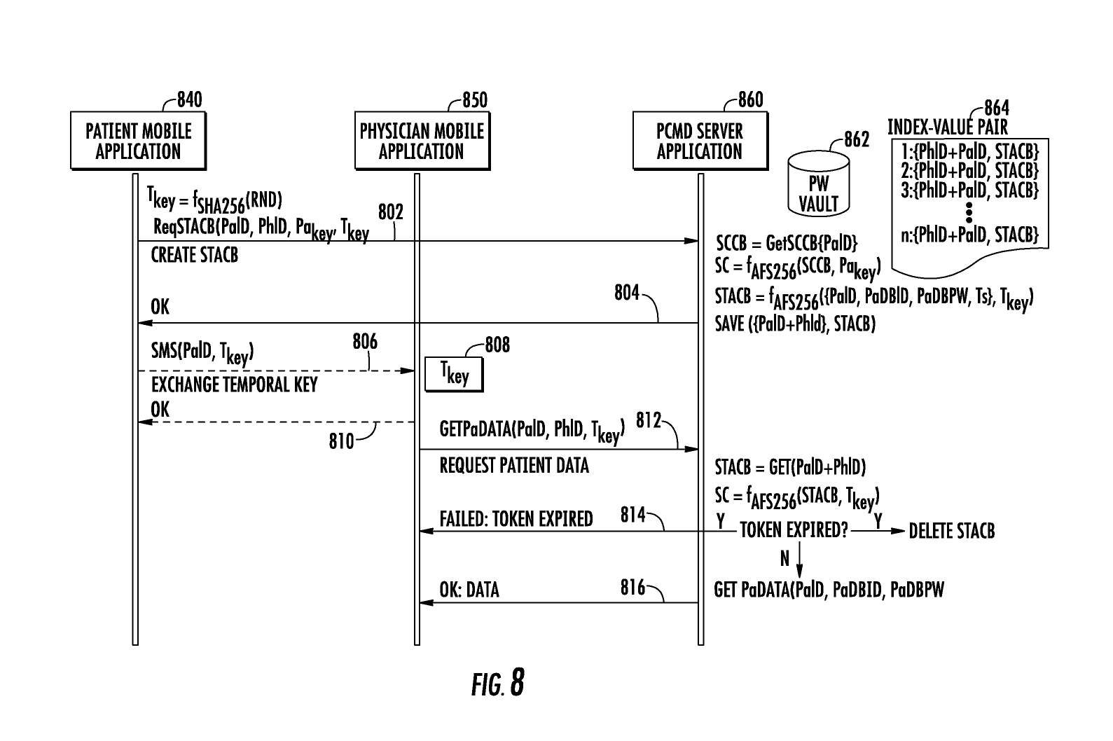

FIG. 8 illustrates a token PCMD sequence diagram that highlights the patient/physician temporal token generation and use sequence described above with reference to FIG. 7. The operations depicted in FIG. 8 may performed by a patient mobile application 840 (e.g., a patient PCMD application executing on a patient device), a physician mobile application (e.g., a physician PCMD application executing on a physician device), a PCMD server application 860 (e.g., a PCMD application executing on a PCMD server), a password vault 862, an index value database 864. For example, the patient mobile application 840 may transmit a request 802 to the PCMD server application 860. PCMD server application 860 may receive and process the request 802 and transmit a response 804 (e.g., an acknowledgement or ACK signal) to the patient mobile application 840. The patient mobile application 840 may transmit a message 806 to the physician mobile application 850. The physician mobile application 850 may receive and process the message 806, generate a temporal key (T_{key}) 808, and transmit a response 810 (e.g., an acknowledgement or ACK signal) to the patient mobile application 840. The physician mobile application 850 may transmit a request 812 to the PCMD server application 860. PCMD server application 860 may receive and process the request 812 and transmit a response 816 (e.g., an acknowledgement or ACK signal and the requested patient data) to the physician mobile application 850.

### Web Security Summary

In some embodiments, the concept of a SCCB and STACB provides an innovative way to securely access patient and physician data. Both SCCB and STACB use the concept of secret keys that are not stored on the PCMD server. The keys are made securely available to the PCMD server only when data needs to be retrieved. The PCMD server also deletes all remnants of keys after each HTTPS session.

### Patient PCMD Application

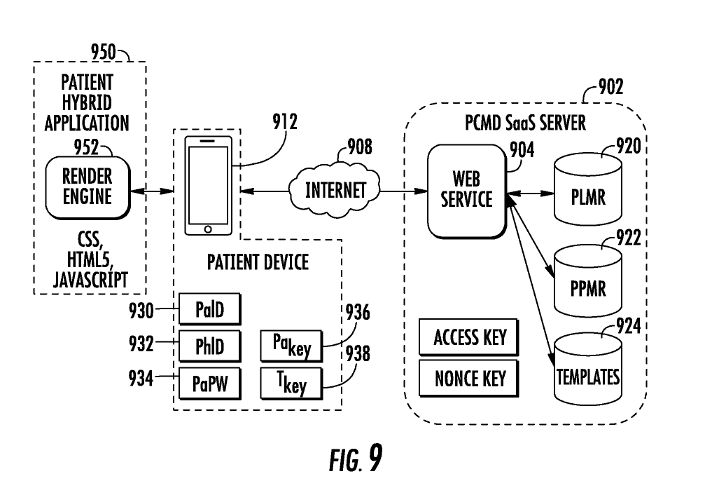

In some embodiments, the patient PCMD application may be a mobile hybrid application configured to view, add, edit, update, and manage the patient's medical information and records (e.g., PLMRs and PPMRs) and to access security and user interface features. In one example, the patient PCMD application is primarily a viewer and editor of the patient's PPMR and PLMR. The patient PCMD application architecture is highlighted in FIG. 9.

FIG. 9 illustrates a system diagram of a set of devices that may be involved in providing a patient PCMD application in accordance with some example embodiments described herein. As shown in FIG. 9, the patient PCMD application 950 comprise a render engine 952. The patient PCMD application 950 may comprise computer-executable program code comprising program code instructions stored in a memory of the patient device 912 and configured to be executed by the processing circuitry of the patient device 912. The patient device 912 may comprise PaID 930, PhD 932, PaPW 934, Pa_{key} 936, and T_{key} 938. The patient device 912 may be in communication with the PCMD server 902 through a communications network 908 (e.g., the Internet). The PCMD server 902 may be a SaaS server and comprise a web service 904, PLMR database 920, PPMR database 922, and templates database 924 (e.g., comprising PLMTs and PPMTs). The PCMD server 902 may also comprise an access key and a nonce key.

In some embodiments, the patient PCMD application 950 may be a hybrid application. One advantage of a hybrid application is that the programmer only has to write the application once for multiple mobile device operating systems (e.g., iOS, Android, Windows). Accordingly, a hybrid application provides a robust user UX and multi-platform interoperability.

In some embodiments, the patient PCMD application 950 may implement a platform specific native WebView, which may be similar to a web browser. The WebView UX may provide a similar look and feel of the mobile device operating system. For example, the patient PCMD application 950, when running on an iOS or Android operating system, has the same visual properties as a native application. In some instances, the WebView UX may use CSS, HTML5 and JavaScript as the application language.

In some embodiments, the patient may use the patient PCMD application 950 to view, update, add, and edit data to the patient's PLMR and PPMRs. In one example embodiment, most of the data entered in the PLMR it is permanent and cannot be modified such that the PLMR is a permanent patient life medical record. Some PLMR data may be modified such as the patient's current address, physician name, emergency contacts, and medical alerts that may be needed by emergency medical technicians (EMTs). The patient may use the patient PCMD application 950 to view PPMRs but may only modify certain portions of those PPMRs, such as complaint and illness history.

In some embodiments, the patient PCMD application 950 may use an innovative UX referred to herein as ViewDock. ViewDock allows a patient to select specific views through a subject index. Once the subject is selected the data is presented in a book or chart like format. The patient can then swipe from page to page to locate, view, update, add, and edit data.

In some embodiments, the patient PCMD application 950 directly connects to the PCMD server 902. The connection may be a standard secure HTTPS internet connection for data security. In some embodiments, all connections to the API of the PCMD server 902 may use HMAC security to ensure that the patient has the correct security credentials to access patient data. The HMAC uses the patient's PaID 930 and Pa_{key} 936 as security credentials. The PaID 930 identifies the patient and the Pa_{key} 936 is created by a SHA-256 hash of the concatenation of the {PaID 930+PaPW 934}. The Pa_{key} 936 also may be used to encrypt and decrypt the patient's PPMR and PLMR.

In some embodiments, during the start of an examination, the patient may use the patient PCMD application 950 to input the physician's name to allow the physician access to the patient's medical data. The patient PCMD application 950 creates a temporal token referred to herein as a STACB. In some embodiments, the STACB is used to extract the database security credentials without the need of an open key stored on the server. Next, the patient device 912 generates a random number and uses it to create a temporal key (T_{key}) 938. The T_{key} 938 is used to encrypt the STACB. The encrypted STACB is stored in the PCMD server 902. The T_{key} 938 is then sent to the physician device using, for example, cellular SMS. The use of SMS ensures that only the physician device has a copy of the T_{key} 938. The physician PCMD mobile application uses the T_{key} 938 to access the STACB token on the PCMD server. The token is temporal and is only valid for a predetermined length of time. In one illustrative example, the predetermined length of time may be one hour. Once the token has expired, the token can no longer be used to access the patient's data.

Thus, the PCMD medical architecture is quite secure with a focus on the patient controlling all access to his/her data. Only the patient may encrypt, decrypt, view, add, or edit his/her data. The patient also may provide medical professionals with access to his/her patient data only through the use of a temporal key and secure credentials token.

### Physician PCMD Application

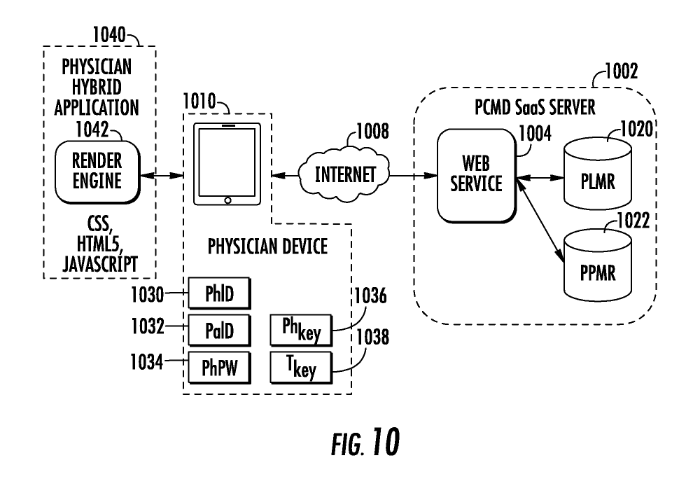

In some embodiments, the physician PCMD application may be a mobile hybrid application configured to view, add, and edit medical information. In one example, the physician PCMD application is primarily a viewer of the patient's PLMR and PPMR and an editor of the PPMR. The physician PCMD application architecture is highlighted in FIG. 10.

FIG. 10 illustrates a system diagram of a set of devices that may be involved in providing a physician PCMD application in accordance with some example embodiments described herein. As shown in FIG. 10, the physician PCMD application 1040 comprise a render engine 1042. The physician PCMD application 1040 may comprise computer-executable program code comprising program code instructions stored in a memory of the physician device 1010 and configured to be executed by the processing circuitry of the physician device 1010. The physician device 1010 may comprise PhID 1030, P1ID 1032, PhPW 1034, Ph_{key} 1036, and T_{key} 1038. The physician device 1010 may be in communication with the PCMD server 1002 through a communications network 1008 (e.g., the Internet). The PCMD server 1002 may be a SaaS server and comprise a web service 1004, PLMR database 1020 and PPMR database 1022.

In some embodiments, the physician PCMD application 1040 may be a hybrid application that is similar in architecture to the patient PCMD application 950 described with reference to FIG. 9. In some instances, the physician PCMD application 1040 may differ from the patient PCMD application 950 in that the physician PCMD application 1040. The physician PCMD application 1040 may utilize the ViewDock viewing interface. ViewDock may allow the physician to select a specific patient topic by the use of an index. Once a topic is selected, the physician may scan through the information like a book or chart.

In some embodiments, the physician PCMD application 1040 may connect to the PCMD server 1002 via communications network 1008. The physician PCMD application 1040 may wait for the patient device to connect to the patient PCMD application. The physician device may receive a notification from the patient device through the exchange of a temporal encryption key using cellular SMS. The temporal key may allow the physician to view the patients' medical data. The key is temporal and only is valid for a predetermined length of time (e.g., one hour). Once the token has expired, the physician device may no longer view the patient data. If the physician needs to continue to view patient data (e.g., during a medical examination or procedure that lasts longer than one hour), the patient device may generate a new random temporal key and transmit that key to the physician device 1010 via SMS.

In some embodiments, the physician may proceed with the examination and, using the physician PCMD application 1040, update the PPMT as the examination progresses. The physician may also use the physician PCMD application 1040 to refer to the patient's PLMR for past medical history or family medical history. The physician may use the physician PCMD application 1040 to enter the examination, test, diagnosis, and treatment recommendations. This information may be entered as text, video, audio (e.g., dictation), images (e.g., a picture of the patient or a portion of the patient's body), scanned, or downloaded. In some instances, the physician may take a picture of a document (e.g., examination notes, prescriptions, X-rays, CAT scans, EKG printouts, etc.) using the physician device 1010.

In some embodiments, the physician may also use the physician PCMD application 1040 to input account information (e.g., all relevant examination codes) that then are emailed to the physician's office manager for correct charges and billing. In some embodiments, the physician may also use the physician PCMD application 1040 to request additional medical testing. For example, the physician may use the physician PCMD application 1040 to generate a medical test order and then digitally sign the medical test order to assure physician authenticity and that the requests were not tampered with. The physician PCMD application 1040 then may transmit (e.g., via e-mail) the medical test order to the medical test facility. In some embodiments, the physician may also use the physician PCMD application 1040 to issue pharmaceutical prescriptions. For example, the physician may use the physician PCMD application 1040 to input the prescription and digitally sign the prescription to verify authenticity and that the request was not tampered with. The physician PCMD application 1040 then may transmit (e.g., via e-mail) the prescription to the patient's pharmacy (e.g., the patient's current pharmacy as indicated in the patient's PLMR or PPMR).

In some embodiments, the PCMD server 902 may store any or all of the above information as metadata (e.g., in the PLMR database 1020, the PPMR database 1022, or both). Once the physician has finished adding patient data to the PPMT and generated the PPMR (and, in some instances, transmitted a request from the physician device 1010 to store the PPMT, the patient data provided in response to the PPMT, the PPMR, or a combination thereof), the PCMD server 902 may add the PPMR to the patient's PLMR in the patient's database (e.g., PLMR database 1020). The PCMD server 902 also may save a copy of the PPMR in the physician's database (e.g., PPMR database 1022) for future reference and administration. The physician's database may be secured by the Ph_{key} and thus only viewable by the physician.

### Web Targeted Medical Marketing

In some embodiments, the PCMD applications disclosed herein also may integrates patient web marketing. The PCMD system may collect metadata about the patient and store that patient metadata in the physician's database. The PCMD server may analyze the patient metadata and generated targeted medical advertising information to the patient, the physician, or both that might be useful for the patient. This medical advertising information may comprise data or electronic information comprising, for example, mobile advertising, identity marketing, pharmaceutical information, medical supplies, insurance information, patient life benefits, physician announcements, and patient reminders.

In some embodiments, the PCMD server may receive information and advertisements from a multitude of medical services, pharmaceutical manufacturers, medical suppliers, and insurance companies. In one illustrative example, the PCMD server may receive information about new medicines or medical procedures that might benefit the patient. In another illustrative example, the PCMD server may receive announcements about a sale on certain medical equipment and supplies associated with the patient's medical condition. In yet another illustrative example, the PCMD server may generate and transmit to the patient device helpful information related to the patient's ailment. This information may be provided as a public service and help improve the patient's quality of life. In some embodiments, the physician may also use this feature to remind patients about upcoming appointments and test results, new services, hours, vacations, and openings in the calendar for potential appointments. According, the PCMD system may provide a helpful tool for the physician to keep in touch with his/her patients.

In some embodiments, the PCMD server may match the data with the patient's metadata and transmit the information to the patient PCMD application. The patient may choose to review or discard this information. If the patient reviews the information, a PCMD advertisement credit may be generated and recorded. Subsequently, these PCMD advertisement credits may be used to charge advertisement fees to respective companies.

### Telemedicine

In some embodiments, the PCMD application disclosed herein may be a fully Internet integrated client/server application. The PCMD application may have an integrated telemedicine service that allows the patient and physician to connect remotely using both voice and video. Utilizing the PCMD server as a connection point, both the physician and patient may discuss and review medical issues and potential treatment using physician and patient PCMD applications executing on their respective devices. The patient also may share his/her PLMR and PPMR with the physician using the patient PCMD application. The physician may review the data using the physician PCMD application, discuss the patient's complaint and history, and even make observations using the imaging features (e.g., camera) of the physician device. The physician may then diagnose and formulate a treatment plan for the patient. The physician may even order additional tests or prescriptions remotely using the PCMD secure digital signature facilities. The physician (or, in some instances, the patient) may record all impressions, tests, prescriptions, and recommendations in the PPMT and save the completed PPMT as a PPMR in physician's database and the patient's database.

### Server-Based PCMD Summary

In conventional systems, patient data is centrally managed by physicians, medical associations, hospitals, and test centers. The patient's medical data is often scattered and not readily accessible by patients or physicians. Although there has been some progress towards centralizing patient data, all of that progress is based on association centric medical data (ACMD) systems. In these ACMD systems, patient data is available to the association medical professionals only if the patient is in the association. The patient data is no longer available once the patient is outside the association. Patients and physicians need easier access to critical patient medical data.

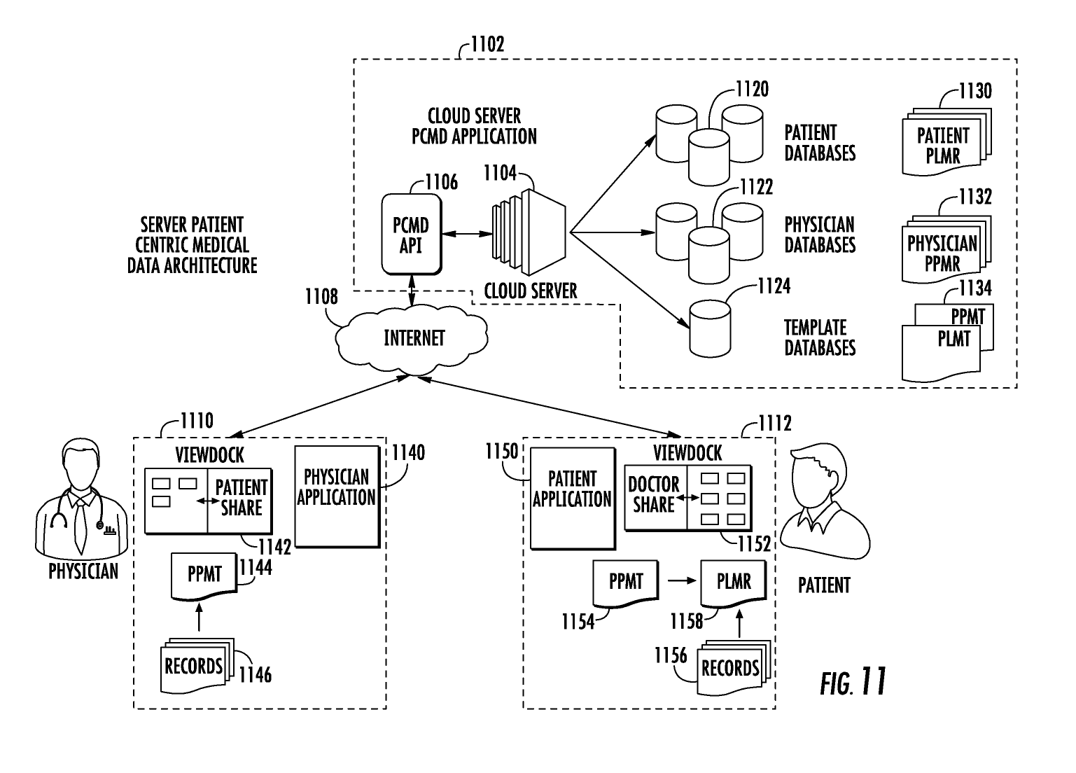

In contrast to these conventional systems, the PCMD system described herein provides improved access to patient data using a patient centric medical data system. An illustrative PCMD system architecture is highlighted in FIG. 11.

FIG. 11 illustrates a system diagram of a set of devices that may be involved in providing a server-based PCMD application architecture in accordance with some example embodiments described herein. As shown in FIG. 11, the server-based PCMD architecture may comprise a PCMD server 1102 in communication with a physician device 1110 and a patient device 1112 through a communications network 1108 (e.g., the Internet). The PCMD server 1102 may comprise a cloud server-based PCMD application, a cloud server 1104, a PCMD API 1106, one or more patient databases 1120, one or more physician databases 1122, and one or more template databases 1124. The one or more patient databases 1120 may comprise, for example, one or more PLMRs 1130. The one or more physician databases 1122 may comprise, for example, one or more PPMRs 1132. The one or more template databases 1124 may comprise, for example, one or more templates such as PLMTs and PPMTs. The physician device 1110 may comprise a physician PCMD application 1140, a mobile viewing application 1142 (e.g., ViewDock), a PPMT 1144, and one or more records 1146 for one or more patients. The patient device 1112 may comprise a patient PCMD application 1150, a mobile viewing application 1152 (e.g., ViewDock), a PPMT 1154, a PLMR 1158, and one or more records 1156.

FIGS. 1-12 thus illustrate a server-based PCMD architecture in which the patient's medical data is centrally managed in a cloud-based PCMD server rather than being scatter across multiple doctors, hospitals, and test facilities. In some embodiments, the patient may be responsible for collecting and managing all of the data. The PCMD server may be highly secure using the innovative security technologies disclosed herein. Patient data may be conveniently accessed using a mobile PCMD application. The PCMD application allows the patient to collect, edit and view his/her data. Medical professionals may also add, view and edit data with permission from the patient. The data is accessible from the Internet and may be viewed anywhere and at any time. The PCMD system allows patients to always be in control of their own medical data. The PCMD system also may make patients' medical data readily available to medical professionals at anytime, anywhere in the world. The PCMD system thus makes access to patient data ubiquitous.

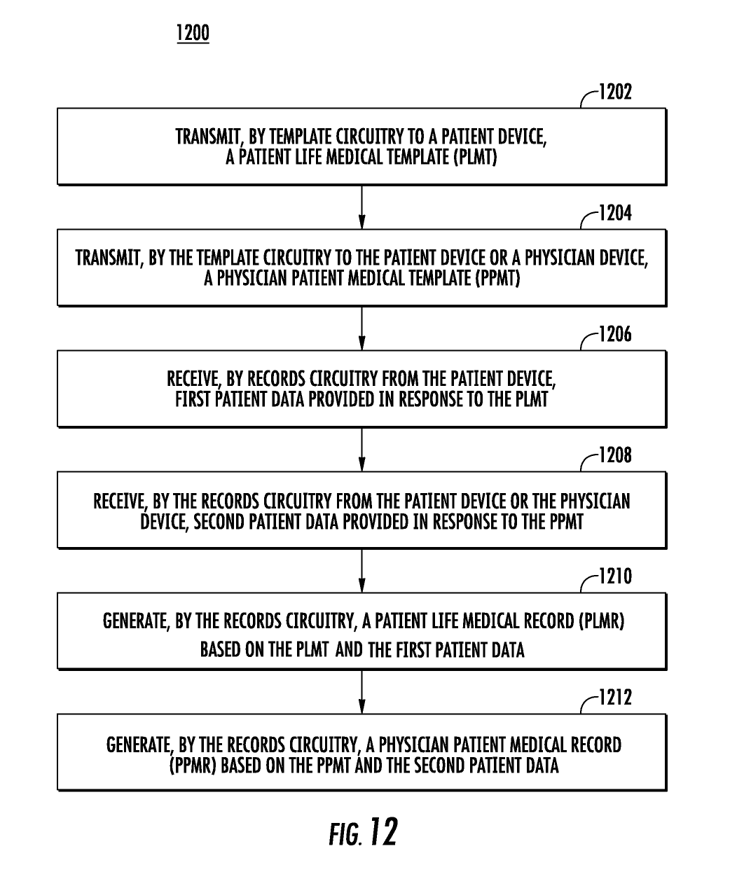

Having described specific components of example devices involved in the present disclosure, example procedures for managing server-based PCMD are described below in connection with FIG. 12.

### Example Operations for Managing Server-Based PCMD

Turning to FIG. 12, an example flowchart 1200 is illustrated that contains example operations for managing server-based PCMD according to an example embodiment. The operations illustrated in FIG. 12 may, for example, be performed by one or more components described with reference to PCMD system 102 shown in FIG. 1, by a physician device 110 or a patient device 112 in communication with PCMD system 102, by apparatus 200 shown in FIG. 2, or by any combination thereof. In some embodiments, the various operations described in connection with FIG. 12 may be performed by the apparatus 200 by or through the use of one or more of processing circuitry 202, memory 204, input-output circuitry 206, communications circuitry 208, template circuitry 210, records circuitry 212, STT circuitry 214, NLP circuitry 216, TTS circuitry 218, sensor circuitry 220, accounting circuitry 222, appointment circuitry 224, medical test circuitry 226, pharmaceutical circuitry 228, UI circuitry 230, and communications security circuitry 232, any other suitable circuitry, and any combination thereof.

As shown by operation 1202, the apparatus 200 includes means, such as template circuitry or the like, for transmitting a first template, such as a PLMT, to a first computing device, such as a patient device. The template circuitry may be any suitable template circuitry described herein, such as template circuitry 210 described with reference to FIG. 2. In some embodiments, the apparatus 200 may include means, such as communications circuitry in communication with the template circuitry or the like, for transmitting the first template to the patient device (e.g., patient device 112). The communications circuitry may be any suitable communications circuitry described herein, such as communications circuitry 208 described with reference to FIG. 2. For example, the template circuitry may transmit the first template to a patient device 112 for visual output via input-output circuitry, user interface circuitry, or both comprised by the patient device 112. In another example, the apparatus 200 may transmit the first template to NLP circuitry (e.g., NLP circuitry 216), TTS circuitry (e.g., TTS circuitry 218), or the like for producing an audio output of a vocal representation of the first template via input-output circuitry 206.

As shown by operation 1204, the apparatus 200 includes means, such as the template circuitry or the like, for transmitting a second template, such as a PPMT, to a second computing device, such as the patient device or a physician device. In some embodiments, the apparatus 200 may include means, such as communications circuitry in communication with the template circuitry or the like, for transmitting the second template to the patient device (e.g., patient device 112) or the physician device (e.g., physician device 110). For example, the template circuitry may transmit the second template to a patient device 112 or a physician device 110 for visual output via input-output circuitry, user interface circuitry, or both comprised by the patient device 112 or the physician device 110. In another example, the apparatus 200 may transmit the second template to TTS circuitry or the like for producing an audio output of a vocal representation of the second template via input-output circuitry 206.

As shown by operation 1206, the apparatus 200 includes means, such as records circuitry or the like, for receiving first patient data from the patient device. The first patient data may be provided by a user of the patient device in response to the transmission of the first template. The records circuitry may be any suitable records circuitry described herein, such as records circuitry 212 described with reference to FIG. 2. In some embodiments, the apparatus 200 may include means, such as communications circuitry in communication with the records circuitry or the like, for receiving the first patient data from the patient device (e.g., patient device 112). The communications circuitry may be any suitable communications circuitry described herein, such as communications circuitry 208 described with reference to FIG. 2. For example, the records circuitry may receive the first patient data from a patient device 112. The received first patient data may have been provided by a user of patient device 112 via input-output circuitry, user interface circuitry, or both comprised by the patient device 112. In another example, the apparatus 200 may receive the first patient data from a medical data source server (e.g., medical data source device 114). In yet another example, the apparatus 200 may receive the first patient data from STT circuitry (e.g., STT circuitry 214), NLP circuitry (e.g., NLP circuitry 216), or the like, such as when the first patient data was provided by the user of the patient device in speech or video format.

As shown by operation 1208, the apparatus 200 includes means, such as records circuitry or the like, for receiving second patient data from the patient device or the physician device. The second patient data may be provided by a user of the patient device or physician device in response to the transmission of the second template. In some embodiments, the apparatus 200 may include means, such as communications circuitry in communication with the records circuitry or the like, for receiving the second patient data from the patient device (e.g., patient device 112) or the physician device (e.g., physician device 110). For example, the records circuitry may receive the second patient data from a patient device 112 or a physician device 110. The received second patient data may have been provided by a user of patient device 112 or physician device 110 via input-output circuitry, user interface circuitry, or both comprised by the patient device 112 or the physician device 110. In another example, the apparatus 200 may receive the second patient data from a medical data source server (e.g., medical data source device 114). In yet another example, the apparatus 200 may receive the second patient data from STT circuitry (e.g., STT circuitry 214), NLP circuitry (e.g., NLP circuitry 216), or the like, such as when the second patient data was provided by the user of the patient device or the physician device in speech or video format.

As shown by operation 1210, the apparatus 200 includes means, such as the records circuitry or the like, for generating a first record, such as a PLMR, based on the first template and the first patient data. For example, the apparatus 200 may comprise records circuitry 212 for generating a PLMR based on the PLMT, the first patient data provided by a patient in response to the PLMT. In some embodiments, the records circuitry 212 may store the first record (e.g., PLMR) in a first database server (e.g., the patient's database server or cloud account) accessible by the first computing device.

As shown by operation 1212, the apparatus 200 includes means, such as the records circuitry or the like, for generating a second record, such as a PPMR, based on the second template and the second patient data. For example, the apparatus 200 may comprise records circuitry 212 for generating a PPMR based on the PPMT and the second patient data provided by the patient or a physician in response to the PPMT. In some embodiments, the records circuitry 212 may store the second record (e.g., PPMR) in a second database server (e.g., the physician's database server or cloud account; the patient's database server or cloud account) accessible by the second computing device.

In some embodiments, operations 1202, 1204, 1206, 1208, 1210, and 1212 may not necessarily occur in the order depicted in FIG. 12, and in some cases one or more of the operations depicted in FIG. 12 may occur substantially simultaneously, or additional steps may be involved before, after, or between any of the operations shown in FIG. 12. Moreover, although FIG. 12 illustrates operations 1204 and 1208 as occurring after operation 1202, this is for ease of explanation only, and multiple embodiments are contemplated here. For instance, in some embodiments, operation 1204 may occur prior to performance of operation 1202, and operation 1208 may occur prior to performance of operation 1206. In other embodiments, operations 1202 and 1206 need not occur at all (and this may be by design in some embodiments, and in some embodiments this may be because the PLMT and first patient data has already been received by the PCMD system described herein), and instead the procedure advances directly from operation 1204 to operation 1208.

FIG. 12 thus illustrates a flowchart describing the operation of various systems (e.g., PCMD system 102 described with reference to FIG. 1), apparatuses (e.g., apparatus 200 described with reference to FIG. 2), methods, and computer program products according to example embodiments contemplated herein. It will be understood that each operation of the flowchart, and combinations of operations in the flowchart, may be implemented by various means, such as hardware, firmware, processor, circuitry, and/or other devices associated with execution of software including one or more computer program instructions. For example, one or more of the procedures described above may be performed by execution of computer program instructions. In this regard, the computer program instructions that, when executed, cause performance of the procedures described above may be stored by a memory (e.g., memory 204) of an apparatus (e.g., apparatus 200) and executed by a processor (e.g., processing circuitry 202) of the apparatus. As will be appreciated, any such computer program instructions may be loaded onto a computer or other programmable apparatus (e.g., hardware) to produce a machine, such that the resulting computer or other programmable apparatus implements the functions specified in the flowchart operations. These computer program instructions may also be stored in a computer-readable memory that may direct a computer or other programmable apparatus to function in a particular manner, such that the instructions stored in the computer-readable memory produce an article of manufacture, the execution of which implements the functions specified in the flowchart operations. The computer program instructions may also be loaded onto a computer or other programmable apparatus to cause a series of operations to be performed on the computer or other programmable apparatus to produce a computer-implemented process such that the instructions executed on the computer or other programmable apparatus provide operations for implementing the functions specified in the flowchart operations.

The flowchart operations described with reference to FIG. 12 support combinations of means for performing the specified functions and combinations of operations for performing the specified functions. It will be understood that one or more operations of the flowchart, and combinations of operations in the flowchart, can be implemented by special purpose hardware-based computer systems which perform the specified functions, or combinations of special purpose hardware and computer instructions.

### CONCLUSION

While various embodiments in accordance with the principles disclosed herein have been shown and described above, modifications thereof may be made by one skilled in the art without departing from the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow, that scope including all equivalents of the subject matter of the claims. Each and every claim is incorporated as further disclosure into the specification and the claims are embodiment(s) of the present disclosure. Furthermore, any advantages and features described above may relate to specific embodiments, but shall not limit the application of such issued claims to processes and structures accomplishing any or all of the above advantages or having any or all of the above features.

In addition, the section headings used herein are provided for consistency with the suggestions under 37 C.F.R. 1.77 or to otherwise provide organizational cues. These headings shall not limit or characterize the disclosure set out in any claims that may issue from this disclosure. For instance, a description of a technology in the “Background” is not to be construed as an admission that certain technology is prior art to any disclosure in this disclosure. Neither is the “Summary” to be considered as a limiting characterization of the disclosure set forth in issued claims. Furthermore, any reference in this disclosure to “disclosure” or “embodiment” in the singular should not be used to argue that there is only a single point of novelty in this disclosure. Multiple embodiments of the present disclosure may be set forth according to the limitations of the multiple claims issuing from this disclosure, and such claims accordingly define the disclosure, and their equivalents, that are protected thereby. In all instances, the scope of the claims shall be considered on their own merits in light of this disclosure, but should not be constrained by the headings set forth herein.

Also, techniques, systems, subsystems, and methods described and illustrated in the various embodiments as discrete or separate may be combined or integrated with other systems, modules, techniques, or methods without departing from the scope of the present disclosure. Other devices or components shown or discussed as coupled to, or in communication with, each other may be indirectly coupled through some intermediate device or component, whether electrically, mechanically, or otherwise. Other examples of changes, substitutions, and alterations are ascertainable by one skilled in the art and could be made without departing from the scope disclosed herein.

Many modifications and other embodiments of the disclosure set forth herein will come to mind to one skilled in the art to which these embodiments pertain having the benefit of teachings presented in the foregoing descriptions and the associated figures. Although the figures only show certain components of the apparatus and systems described herein, it is understood that various other components may be used in conjunction with the supply management system. Therefore, it is to be understood that the disclosure is not to be limited to the specific embodiments disclosed and that modifications and other embodiments are intended to be included within the scope of the appended claims. For example, the various elements or components may be combined, rearranged, or integrated in another system or certain features may be omitted or not implemented. Moreover, the steps in any method described above may not necessarily occur in the order depicted in the accompanying figures, and in some cases one or more of the steps depicted may occur substantially simultaneously, or additional steps may be involved. Although specific terms are employed herein, they are used in a generic and descriptive sense only and not for purposes of limitation.

What is claimed is:

1. A system for managing patient centric medical data (PCMD), the system configured to:
transmit a patient life medical template (PLMT) for display to a first computing device;
receive first patient data for a particular patient provided as input to the PLMT via the first computing device;
generate a first record based on the first patient data provided as input to the PLMT via the first computing device;
transmit a physician patient medical template (PPMT) for display to a second computing device;
receive second patient data for the particular patient provided as input to the PPMT via the second computing device; and
generate a second record based on the second patient data provided as input to the PPMT via the second computing device.

2. The system of claim 1, wherein the first computing device comprises a patient device.

3. The system of claim 2, wherein the second computing device comprises a physician device.

4. The system of claim 1, wherein the system is further configured to:
store the first record in a first database server accessible by the first computing device; and
store the second record in a second database server accessible by the second computing device.

5. The system of claim 1, wherein the system is further configured to generate accounting information and transmit the generated accounting information.

6. The system of claim 1, wherein the system is further configured to generate appointment information and transmit the generated appointment information.

7. The system of claim 1, wherein the system is further configured to generate medical test information and transmit the generated medical test information.

8. The system of claim 1, wherein the system is further configured to generate pharmaceutical information and transmit the generated pharmaceutical test information.

9. The system of claim 1, wherein the first computing device is executing a first mobile viewing application to view the PLMT and the second computing device is executing a second mobile viewing application to view the PPMT.

10. The system of claim 1, wherein the system is further configured to provide a secure credential cipher block (SCCB) application.

11. The system of claim 1, wherein the system is further configured to provide a secure temporal access cipher block (STACB) application.

12. A method for managing patient centric medical data (PCMD), the method comprising:
transmitting, by processing circuitry, a patient life medical template (PLMT) for display to a first computing device;
receiving, by the processing circuitry, first patient data for a particular patient provided as input to the PLMT via the first computing device;
generating, by the processing circuitry, a first record based on the first patient data provided as input to the PLMT via the first computing device;
transmitting, by the processing circuitry, a physician patient medical template (PPMT) for display to a second computing device;
receiving, by the processing circuitry, second patient data for the particular patient provided as input to the PPMT via the second computing device; and
generating, by the processing circuitry, a second record based on the second patient data provided as input to the PPMT via the second computing device.

13. The method of claim 12, wherein the first computing device comprises a patient device.

14. The method of claim 12, wherein the second computing device comprises a physician device.

15. The method of claim 12, wherein the system is further configured to provide a secure credential cipher block (SCCB) application.

16. The method of claim 12, wherein the system is further configured to provide a secure temporal access cipher block (STACB) application.

17. A computer program product for managing patient centric medical data (PCMD), the computer program product comprising at least one non-transitory computer-readable storage medium storing program instructions that, when executed, cause a server to:
transmit a patient life medical template (PLMT) for display to a first computing device;
receive first patient data for a particular patient provided as input to the PLMT via the first computing device;
generate a first record based on the first patient data provided as input to the PLMT via the first computing device;
transmit a physician patient medical template (PPMT) for display to a second computing device;
receive second patient data for the particular patient provided as input to the PPMT via the second computing device; and
generate a second record based on the second patient data provided as input to the PPMT via the second computing device.

18. The computer program product of claim 17, wherein the first computing device comprises a patient device and the second computing device comprises a physician device.

19. The computer program product of claim 12, wherein the program instructions, when executed, further cause the server to provide a secure credential cipher block (SCCB) application.

20. The computer program product of claim 12, wherein the program instructions, when executed, further cause the server to provide a secure temporal access cipher block (STACB) application.
